(19) United States
(12) Patent Application Publication
(10) Pub. No.: US 2023/0355388 A1
(43) Pub. Date: Nov. 9, 2023

(54) DELIVERY APPARATUS AND METHODS FOR PROSTHETIC VALVE DOCKING DEVICES

(71) Applicant: EDWARDS LIFESCIENCES CORPORATION, Irvine, CA (US)

(72) Inventor: Yuanlong Du, Irvine, CA (US)

(21) Appl. No.: 18/354,295

(22) Filed: Jul. 18, 2023

Related U.S. Application Data
(63) Continuation of application No. PCT/US2022/012775, filed on Jan. 18, 2022.
(60) Provisional application No. 63/138,910, filed on Jan. 19, 2021.

(51) Int. Cl. A61F 2/24

(52) U.S. Cl. CPC A61F 2/2436; A61B 2090/3966

(57) ABSTRACT

A delivery apparatus includes a dock sleeve having a body portion and a tip portion located at a distal end of the body portion and configured to be axially movable relative to a docking device. The body portion includes a lumen configured to receive the docking device therein. The tip portion one or more flaps that are movable between a radially collapsed state and a radially expanded state. In the radially collapsed state, the one or more flaps cover a distal end of the docking device and confine the docking device within the lumen of the body portion. In the radially expanded state, the one or more flaps allow the distal end of the docking device to extend distally from the lumen of the body portion and beyond the tip portion such that the distal end of the docking device is uncovered by the dock sleeve.

### CROSS-REFERENCE TO RELATED APPLICATION

This application is a continuation of PCT patent application no. PCT/US2022/012775, filed on Jan. 18, 2022, which claims the benefit of U.S. Provisional Application No. 63/138,910 filed Jan. 19, 2021, each of which is incorporated herein in its entirety by this specific reference.

### FIELD

The present disclosure is directed to heart valve repair devices and more particularly to delivery apparatus and methods for implanting prosthetic heart valve docking devices.

### BACKGROUND

Prosthetic valves can be used to treat cardiac valvular disorders. Native heart valves (e.g., the aortic, pulmonary, tricuspid and mitral valves) function to prevent backward flow or regurgitation, while allowing forward flow. These heart valves can be rendered less effective by congenital, inflammatory, infectious conditions, etc. Such conditions can eventually lead to serious cardiovascular compromise or death. For many years, the doctors attempted to treat such disorders with surgical repair or replacement of the valve during open heart surgery.

A transcatheter technique for introducing and implanting a prosthetic heart valve using a catheter in a manner that is less invasive than open heart surgery can reduce complications associated with open heart surgery. In this technique, a prosthetic valve can be mounted in a compressed state on the end portion of a catheter and advanced through a blood vessel of the patient until the valve reaches the implantation site. The valve at the catheter tip can then be expanded to its functional size at the site of the defective native valve, such as by inflating a balloon on which the valve is mounted or, for example, the valve can have a resilient, self-expanding stent or frame that expands the valve to its functional size when it is advanced from a delivery sheath at the distal end of the catheter. Optionally, the valve can have a balloon-expandable, self-expanding, mechanically expandable frame, and/or a frame expandable in multiple or a combination of ways.

In some instances, a transcatheter heart valve (THV) may be appropriately sized to be placed inside a particular native valve (e.g., a native aortic valve). As such, the THV may not be suitable for implantation at another native valve (e.g., a native mitral valve) and/or in a patient with a larger native valve. Additionally, or alternatively, the native tissue at the implantation site may not provide sufficient structure for the THV to be secured in place relative to the native tissue. Accordingly, improvements to THVs and the associated transcatheter delivery apparatus are desirable.

### SUMMARY

The present disclosure relates to methods and systems for treating valvular regurgitation and/or other valve issues. Specifically, the present disclosure is directed to a docking device configured to receive a prosthetic valve and the methods of assembling the docking device and implanting the docking device.

Certain examples of the disclosure concern a delivery apparatus. The delivery apparatus can include a dock sleeve having a body portion and a tip portion located at a distal end of the body portion and configured to be axially movable relative to a docking device for a prosthetic implant. The body portion can include a lumen configured to receive the docking device therein. The tip portion can include one or more slits defining one or more flaps. The one or more flaps can be movable between a radially collapsed state and a radially expanded state. In the radially collapsed state, the one or more flaps can cover a distal end of the docking device and occlude the lumen of the body portion. In the radially expanded state, the one or more flaps can allow the distal end of the docking device to extend distally from the lumen of the body portion and beyond the tip portion such that the distal end of the docking device is uncovered by the dock sleeve.

Certain examples of the disclosure concern a dock sleeve for a delivery apparatus configured to implant a docking device. The dock sleeve can include a body portion and a tip portion located at a distal end of the body portion. The dock sleeve can be configured to be axially movable relative to the docking device. The body portion can be configured to cover at least a distal portion of the docking device when the distal end of the body portion axially aligns with a distal end of the docking device. The tip portion can be movable between a radially collapsed state and a radially expanded state. When the body portion covers the distal portion of the docking device, the tip portion in the radially collapsed state can cover the distal end of the docking device, and the tip portion in the radially expanded state can allow the distal end of the docking device to move distally relative to the distal end of the body portion.

Certain examples of the disclosure concern another dock sleeve for implanting a docking device at a native valve. The dock sleeve can include a body portion and a tip portion located at a distal end of the body portion. The dock sleeve can be configured to be axially movable relative to the docking device. The body portion can be configured to cover at least a distal portion of the docking device when the distal end of the body portion axially aligns with a distal end of the docking device. The tip portion can include one or more slits dividing the tip portion into one or more flaps. When the body portion covers the distal portion of the docking device, the one or more flaps can collapse radially inwardly so as to cover the distal end of the docking device and can expand radially outwardly when the distal end of the docking device is advanced distally through the tip portion.

Certain examples of the disclosure also concern an implant assembly. The implant assembly can include a docking device configured to be implanted at a native annulus of a patient, and a dock sleeve including a body portion and a tip portion located at a distal end of the body portion. The dock sleeve can be configured to cover the docking device during one or more portions of a delivery procedure and to be axially movable relative to the docking device such that the docking device can be exposed from the dock sleeve. The body portion can be configured to cover at least a distal portion of the docking device when the distal end of the body portion axially aligns with a distal end of the docking device. The tip portion can be movable between a radially collapsed state and a radially expanded state. When the distal end of the body portion is axially aligned with the distal end of the docking device, the tip portion can be in the radially collapsed state, and when the distal end of the docking device is disposed distal to the tip portion, the tip portion can be in the radially expanded state.

Certain examples of the disclosure also concern an implant assembly including a radially expandable and compressible prosthetic valve, a docking device configured to receive the prosthetic valve, and a dock sleeve configured to be axially movable relative to the docking device. The prosthetic valve can be configured to be radially expandable within the docking device. The dock sleeve can have a body portion and a tip portion located at a distal end of the body portion. The body portion can be configured to cover at least a distal portion of the docking device when the distal end of the body portion aligns with a distal end of the docking device. The tip portion can be movable between a radially collapsed state and a radially expanded state. When the body portion covers the distal portion of the docking device, the tip portion in the radially collapsed state can cover the distal end of the docking device, and the tip portion in the radially expanded state can allow the distal end of the docking device to move distally relative to the distal end of the body portion so as to be uncovered by the dock sleeve.

Certain examples of the disclosure also concern an implant assembly including a docking device configured to surround native tissue at an implantation site of a patient, a dock sleeve configured to cover at least a distal portion and a distal end of the docking device when the docking device is delivered to the implantation site and surrounds the native tissue, and a pusher shaft configured to push the docking device in a distal direction relative to the dock sleeve so that a distal end of the dock sleeve is pressed open to allow the distal portion of the docking device to move out of the dock sleeve when retracting the dock sleeve in a proximal direction while holding the pusher shaft steady or pushing the pusher shaft in a distal direction while holding the dock sleeve steady.

Certain examples of the disclosure also concern a delivery apparatus for implanting a docking device at a native valve. The delivery apparatus can include a dock sleeve configured to cover at least a distal portion and a distal end of the docking device when the docking device is delivered to the native valve, and a pusher shaft configured to push the docking device in a distal direction relative to the dock sleeve so that a distal end of the dock sleeve is pressed open to allow the distal end of the docking device to move out of the dock sleeve when retracting the dock sleeve in a proximal direction while holding the pusher shaft steady or pushing the pusher shaft in a distal direction while holding the dock sleeve steady.

Certain examples of the disclosure also concern a dock sleeve for implanting a docking device at a native valve. The dock sleeve can include a body portion and a tip portion located at a distal end of the body portion. The dock sleeve can be configured to be movable between a covered state and an uncovered state. When the dock sleeve is in the covered state, the body portion can cover at least a distal portion of the docking device and the tip portion covers a distal end of the docking device. When the dock sleeve is in the uncovered state, the distal end of the docking device can extend out of the dock sleeve through the tip portion of the dock sleeve.

Certain examples of the disclosure also concern an implant assembly including a docking device configured to be implanted at an implantation site of a patient, and a dock sleeve configured to be movable between a covered state and an uncovered state. When the dock sleeve is in the covered state, the dock sleeve can cover at least a distal portion and a distal end of the docking device. When the dock sleeve is in the uncovered state, at least a distal end of the docking device can extend out of the dock sleeve through the distal end of the dock sleeve.

Certain examples of the disclosure also concern a delivery apparatus for implanting a docking device at a native valve. The delivery apparatus can include a dock sleeve configured to be movable between a covered state and an uncovered state. When the dock sleeve is in the covered state, the dock sleeve can cover at least a distal portion and a distal end of the docking device. When the dock sleeve is in the uncovered state, at least a distal end of the docking device can extend out of the dock sleeve through the distal end of the dock sleeve.

Certain examples of the disclosure also concern a method of creating a dock sleeve configured to hold a docking device. The method can include creating a dock sleeve having a body portion and a tip portion. The tip portion can completely close a distal end of the body portion. The method can further include adding a coating material to the dock sleeve, and creating at least one slit on the tip portion.

Certain examples of the disclosure also concern a method for implanting a docking device at a target implantation site. The method can include deploying the docking device retained within a dock sleeve at the target implantation site. At least a distal portion of the docking device can be covered by a body portion of the dock sleeve and a distal end of the docking device can be covered by a tip portion of the dock sleeve. The tip portion can be located at a distal end of the body portion. The method can further include removing the dock sleeve from the docking device so that the distal portion and the distal end of the docking device are exposed.

Certain examples of the disclosure further concern a method for implanting a prosthetic valve, and the method can include deploying a docking device retained within a dock sleeve at a native valve, wherein at least a distal portion and a distal end of the docking device can be covered by the dock sleeve, removing the dock sleeve from the docking device so that the distal portion and the distal end of the docking device are exposed, and deploying the prosthetic valve within the docking device.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1A is a side perspective view of a docking device in a helical configuration, according to one example.

FIG. 1B is a top view of the docking device depicted in FIG. 1A.

FIG. 1C is a cross-sectional view of the docking device taken along line 1C-1C depicted in FIG. 1B.

FIG. 1D is a cross-sectional view of the docking device taken along the same line as in FIG. 1C, except in FIG. 1D, the docking device is in a substantially straight delivery configuration.

FIG. 1E is a cross-sectional view of the docking device taken along line 1E-1E depicted in FIG. 1B.

FIG. 1F is a cross-sectional view of the docking device taken along the same line as in FIG. 1E, except in FIG. 1F, the docking device is in a substantially straight delivery configuration.

FIG. 2A is a perspective view a prosthetic valve, according to one example.

FIG. 2B is a perspective view of the prosthetic valve of FIG. 2A with an outer cover, according to one example.

FIG. 3A is a perspective view of an exemplary prosthetic implant assembly comprising the docking device depicted in FIG. 1A and the prosthetic valve of FIG. 2B retained within the docking device.

FIG. 3B is a side elevation view of the prosthetic implant assembly of FIG. 3A.

FIG. 4 is a side view of a delivery assembly comprising a delivery apparatus and the docking device of FIG. 1A, according to one example.

FIG. 5A is a side cross-sectional view of a sleeve shaft, according to one example.

FIG. 5B is a side cross-sectional view of a pusher shaft, according to one example.

FIG. 6A is a side cross-sectional view of an assembly comprising the sleeve shaft of FIG. 5A, the pusher shaft of FIG. 5B, and a delivery sheath, wherein the sleeve shaft covers a docking device.

FIG. 6B is a side cross-sectional view of the same assembly of FIG. 6A, except the docking device is uncovered by the sleeve shaft.

FIG. 7 is a schematic cross-sectional view of a distal end portion of a delivery system, showing fluid flow through lumens within the delivery system.

FIG. 8A illustrates a perspective view of an example of a sleeve shaft covering a docking device and extending outside of a delivery sheath of a delivery system.

FIG. 8B illustrates the sleeve shaft surrounding a pusher shaft after deploying the docking device from the delivery system of FIG. 8A and removing the sleeve shaft from the docking device.

FIG. 9 is a side cross-sectional view of a distal end portion of a dock sleeve comprising a body portion and a tip portion, according to another example.

FIGS. 10A-10D are end views of the tip portion of the dock sleeve of FIG. 9, according to various examples.

FIG. 10E is an end view of a tip portion of a dock sleeve, according to another example.

FIGS. 11A-11B are side profiles of a tip portion of a dock sleeve, according to alternative examples.

FIGS. 12A-12C depict various portions of an exemplary procedure for assembling a sleeve shaft.

FIGS. 13-26 depict various portions of an exemplary implantation procedure in which a delivery apparatus comprising the dock sleeve of FIG. 9 is being used to implant the prosthetic implant assembly of FIG. 3A at a native mitral valve location using a transseptal delivery approach.

### DETAILED DESCRIPTION

### General Considerations

It should be understood that the disclosed examples can be adapted to deliver and implant prosthetic devices in any of the native annuluses of the heart (e.g., the pulmonary, mitral, and tricuspid annuluses), and can be used with any of various delivery approaches (e.g., retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient’s body), while distal motion of the device is motion of the device away from the user and toward the implantation site (e.g., into the patient’s body). The terms “longitudinal” and “axial” refer to an axis extending in the proximal and distal directions, unless otherwise expressly defined.

As used herein, the term “approximately” and “about” means the listed value and any value that is within 10% of the listed value. For example, “about 1 mm” means any value between about 0.9 mm and about 1.1 mm, inclusive.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or”, as well as “and” and “or”.

### Introduction to the Disclosed Technology

Disclosed herein are various systems, apparatuses, methods, etc., including anchoring or docking devices, which can be used in conjunction with expandable prosthetic valves at a native valve annulus (e.g., a native mitral and/or tricuspid valve annulus), in order to more securely implant and hold the prosthetic valve at the implant site. Anchoring/docking devices according to examples of the disclosure can, for example, provide a stable anchoring site, landing zone, or implantation zone at the implant site in which prosthetic valves can be expanded or otherwise implanted. Many of the disclosed docking devices comprise a circular or cylindrically-shaped portion, which can (for example) allow a prosthetic heart valve comprising a circular or cylindrically-shaped valve frame or stent to be expanded or otherwise implanted into native locations with naturally circular cross-sectional profiles and/or in native locations with naturally with non-circular cross sections. In addition to providing an anchoring site for the prosthetic valve, the anchoring/docking devices can be sized and shaped to cinch or draw the native valve (e.g., mitral, tricuspid, etc.) anatomy radially inwards. In this manner, one of the main causes of valve regurgitation (e.g., functional mitral regurgitation), specifically enlargement of the heart (e.g., enlargement of the left ventricle, etc.) and/or valve annulus, and consequent stretching out of the native valve (e.g., mitral, etc.) annulus, can be at least partially offset or counteracted. Some examples of the anchoring or docking devices further include features which, for example, are shaped and/or modified to better hold a position or shape of the docking device during and/or after expansion of a prosthetic valve therein. By providing such anchoring or docking devices, replacement valves can be more securely implanted and held at various valve annuluses, including at the mitral valve annulus which does not have a naturally circular cross-section.

In some instances, a docking device can comprise a paravalvular leakage (PVL) guard (also referred to herein as “a guard member”). The PVL guard can, for example, help reduce regurgitation and/or promote tissue ingrowth between the native tissue and the docking device.

Also disclosed herein are various delivery systems, apparatuses, methods, etc., for implanting the docking devices, including various examples of a dock sleeve configured to cover and/or uncover the docking devices during various portions of an implantation procedure. Example methods of assembling the dock sleeve and implanting the prosthetic valve are also disclosed.

### Exemplary Docking Devices

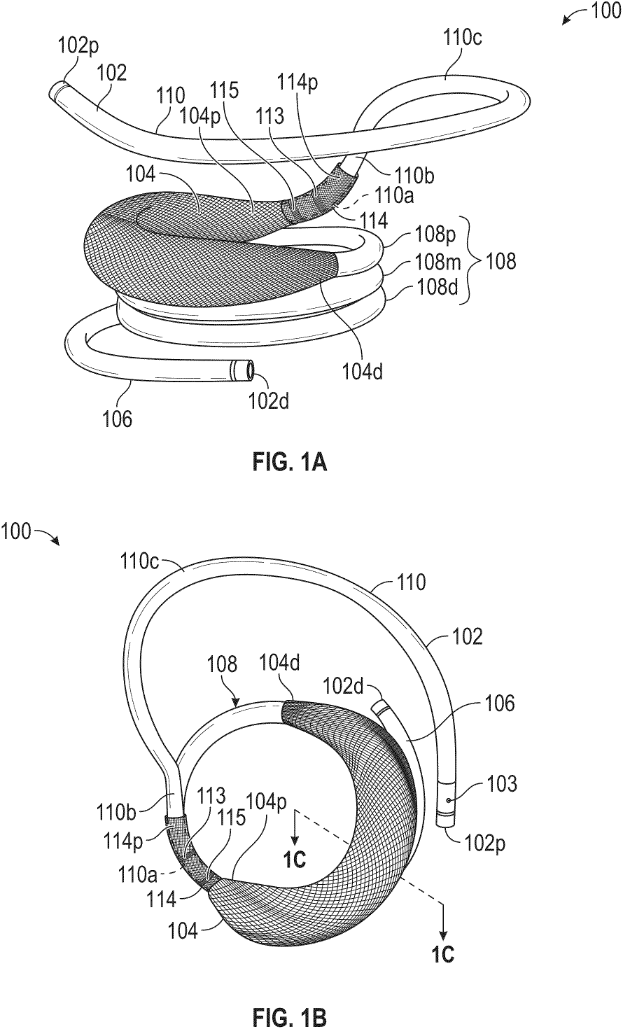

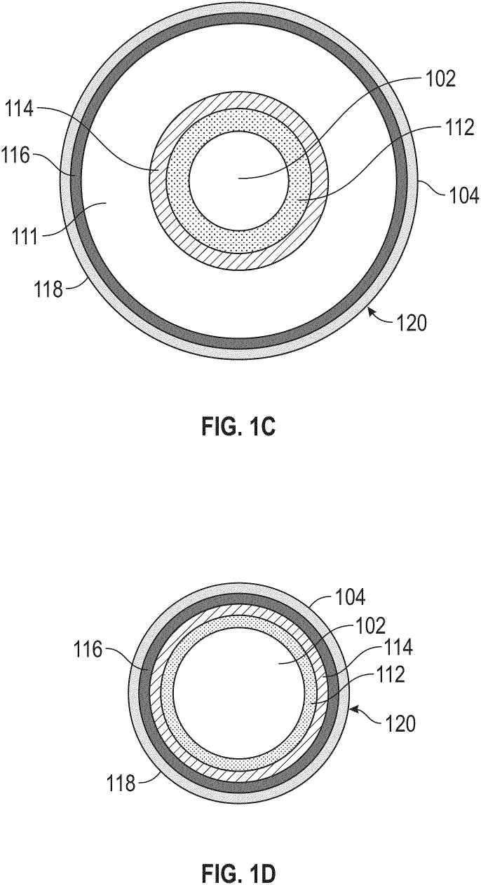

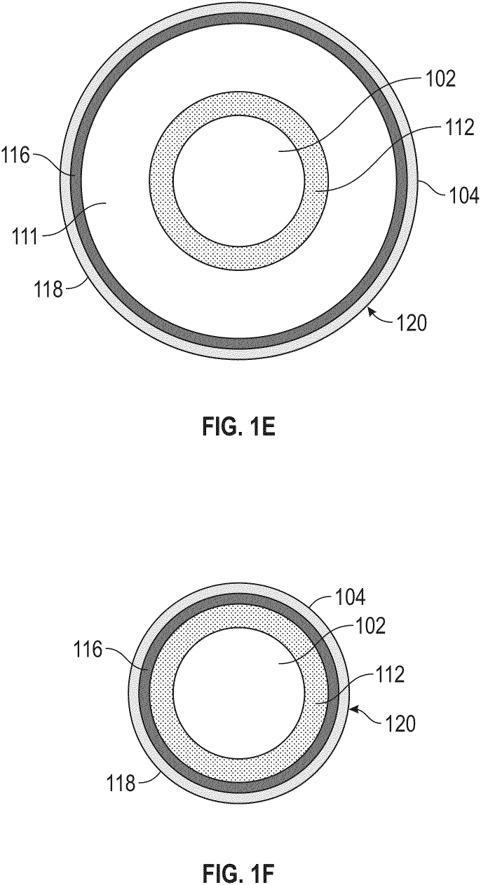

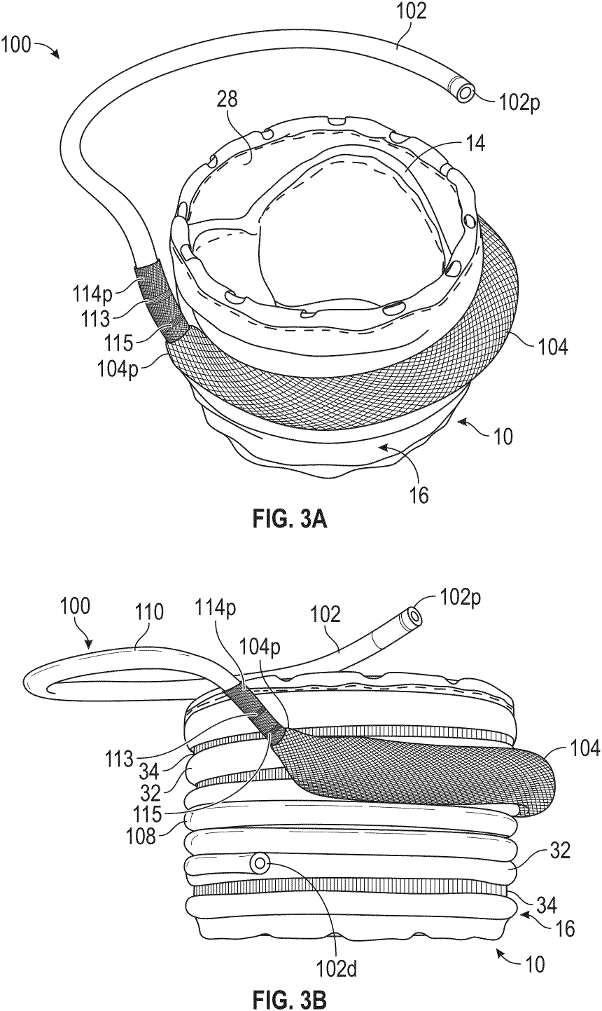

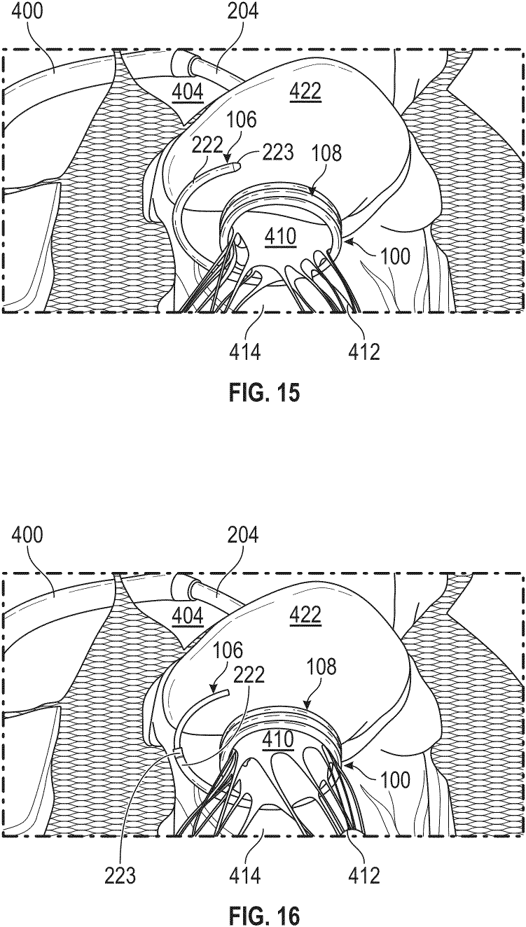

FIGS. 1A-1F show a docking device 100, according to one example. The docking device 100 can, for example, be implanted within a native valve annulus (see, e.g., FIG. 15). As depicted in FIGS. 3A-3B and 26, the docking device can be configured to receive and secure a prosthetic valve within the docking device, thereby securing the prosthetic valve at the native valve annulus.

Referring to FIGS. 1A-1F, the docking device 100 can comprise a coil 102 and a guard member 104 covering at least a portion of the coil 102. In certain examples, the coil 102 can include a shape memory material (e.g., Nitinol) such that the docking device 100 (and the coil 102) can move from a substantially straight configuration (also referred to as “delivery configuration”) when disposed within a delivery sheath of a delivery apparatus (as described more fully below) to a helical configuration (also referred to as “deployed configuration,” as shown in FIGS. 1A-1B) after being removed from the delivery sheath.

In some examples, the docking device 100 can also include a retention element 114 (which in some instances can comprise a braided material) covering at least a portion of the coil 102 and at least being partially covered by the guard member 104. In one example, as illustrated in FIGS. 1A-1B and 3A-3B, at least a proximal end portion 114p of the retention element 114 can extend out of a proximal end of the guard member 104. A plurality of radiopaque markers, e.g., a proximal marker 113 and a distal marker 115, can be placed on the proximal end portion 114p of the braided layer. In another example, the retention element 114 can be completely covered by the guard member 104. The retention element 114 can be designed to interact with the guard member 104 to limit or resist motion of the guard member 104 relative to the coil 102. In addition, the retention element 114 can provide a surface area that encourages or promotes tissue ingrowth, and/or reduce trauma to native tissue.

The coil 102 has a proximal end 102p and a distal end 102d (which also respectively define the proximal and distal ends of the docking device 100). When being disposed within the delivery sheath (e.g., during delivery of the docking device into the vasculature of a patient), a body of the coil 102 between the proximal end 102p and distal end 102d can form a generally straight delivery configuration (i.e., without any coiled or looped portions, but can be flexed or bent) so as to maintain a small radial profile when moving through a patient’s vasculature. After being removed from the delivery sheath and deployed at an implant position, the coil 102 can move from the delivery configuration to the helical deployed configuration and wrap around native tissue adjacent the implant position. For example, when implanting the docking device at the location of a native valve, the coil 102 can be configured to surround native leaflets of the native valve (and the chordae tendineae that connects native leaflets to adjacent papillary muscles, if present), as described further below.

The docking device 100 can be releasably coupled to a delivery apparatus. For example, in certain examples, the docking device 100 can be coupled to a delivery apparatus (as described further below) via a release suture that can be configured to be tied to the docking device 100 and cut for removal. In one example, the release suture can be tied to the docking device 100 through an eyelet or eyehole 103 located adjacent the proximal end 102p of the coil. In another example, the release suture can be tied around a circumferential recess that is located adjacent the proximal end 102p of the coil 102.

In some examples, the docking device 100 in the deployed configuration can be configured to fit at the mitral valve position. In other examples, the docking device can also be shaped and/or adapted for implantation at other native valve positions as well, such as at the tricuspid valve. As described herein, the geometry of the docking device 100 can be configured to engage the native anatomy, which can, for example, provide for increased stability and reduction of relative motion between the docking device 100, the prosthetic valve docked therein, and/or the native anatomy. Reduction of such relative motion can, among other things, prevent material degradation of components of the docking device 100 and/or the prosthetic valve docked therein and/or prevent damage or trauma to the native tissue.

As shown in FIGS. 1A-1B, the coil 102 in the deployed configuration can include a leading turn 106 (or “leading coil”), a central region 108, and stabilization turn 110 (or “stabilization coil”). The central region 108 can possess one or more helical turns having substantially equal inner diameters. The leading turn 106 can extend from a distal end of the central region 108 and has a diameter greater than the diameter of the central region 108 (in one or more configurations). The stabilization turn 110 can extend from a proximal end of the central region 108 and has a diameter greater than the diameter of the central region 108 (in one or more configurations).

In certain examples, the central region 108 can include a plurality of helical turns, such as a proximal turn 108p in connection with the stabilization turn 110, a distal turn 108d in connection with the leading turn 106, and one or more intermediate turns 108m disposed between the proximal turn 108p and the distal turn 108d. In the example shown in FIG. 1A, there is only one intermediate turn 108m between the proximal turn 108p and the distal turn 108d. In other examples, there are more than one intermediate turns 108m between the proximal turn 108p and the distal turn 108d. Some of the helical turns in the central region 108 can be full turns (i.e., rotating 360 degrees). In some examples, the proximal turn 108p and/or the distal turn 108d can be partial turns (e.g., rotating less than 360 degrees, such as 180 degrees, 270 degrees, etc.).

A size of the docking device 100 can be generally selected based on the size of the desired prosthetic valve to be implanted into the patient. In certain examples, the central region 108 can be configured to retain a radially expandable prosthetic valve (as shown in FIGS. 3A-3B and described further below). For example, the inner diameter of the helical turns in the central region 108 can be configured to be smaller than an outer diameter of the prosthetic valve when the prosthetic valve is radially expanded so that additional radial tension can act between the central region 108 and the prosthetic valve to hold the prosthetic valve in place. As described herein, the helical turns (e.g., 108p, 108m, 108d) in the central region 108 are also referred to herein as “functional turns.”

The stabilization turn 110 can be configured to help stabilize the docking device 100 in the desired position. For example, the radial dimension of the stabilization turn 110 can be significantly larger than the radial dimension of the coil in the central region 108, so that the stabilization turn 110 can flare or extend sufficiently outwardly so as to abut or push against the walls of the circulatory system, thereby improving the ability of the docking device 100 to stay in its desired position prior to the implantation of the prosthetic valve. In some examples, the diameter of stabilization turn 110 is desirably larger than the annulus, native valve plane, and atrium for better stabilization. In some examples, the stabilization turn 110 can be a full turn (i.e., rotating about 360 degrees). In some examples, the stabilization turn 110 can be a partial turn (e.g., rotating between about 180 degrees and about 270 degrees).

In one particular example, when implanting the docking device 100 at the native mitral valve location, the functional turns in the central region 108 can be disposed substantially in the left ventricle and the stabilization turn 110 can be disposed substantially in the left atrium. The stabilization turn 110 can be configured to provide one or more points or regions of contact between the docking device 100 and the left atrial wall, such as at least three points of contact in the left atrium or complete contact on the left atrial wall. In certain examples, the points of contact between the docking device 100 and the left atrial wall can form a plane that is approximately parallel to a plane of the native mitral valve.

In some examples, the stabilization turn 110 can have an atrial portion 110a in connection with the proximal turn 108p of the central region 108, a stabilization portion 110c adjacent to the proximal end 102p of the coil 102, and an ascending portion 110b located between the atrial portion 110a and the stabilization portion 110c. Both the atrial portion 110a and the stabilization portion 110c can be generally parallel to the helical turns in the central region 108, whereas the ascending portion 110b can be oriented to be angular relative to the atrial portion 110a and the stabilization portion 110c. The curvature of the stabilization turn 110 can be configured so that the atrial portion 110a and the stabilization portion 110c are disposed on approximately opposite sides when the docking device 100 is fully expanded. When implanting the docking device 100 at the native mitral valve location, the atrial portion 110a can be configured to abut the posterior wall of the left atrium and the stabilization portion 110c can be configured to flare out and press against the anterior wall of the left atrium (see, e.g., FIGS. 18-19 and 26).

As noted above, the leading turn 106 can have a larger radial dimension than the helical turns in the central region 108. As described herein, the leading turn 106 can help more easily guide the coil 102 around and/or through the chordae tendineae geometry and adequately around all native leaflets of the native valve (e.g., the native mitral valve, tricuspid valve, etc.). For example, once the leading turn 106 is navigated around the desired native anatomy, the remaining coil (such as the functional turns) of the docking device 100 can also be guided around the same features. In some examples, the leading turn 106 can be a full turn (i.e., rotating about 360 degrees). In some examples, the leading turn 106 can be a partial turn (e.g., rotating between about 180 degrees and about 270 degrees). As described further below in reference to FIG. 24, when a prosthetic valve is radially expanded within the central region 108 of the coil, the functional turns in the central region 108 can be further radially expanded. As a result, the leading turn 106 can be pulled in the proximal direction and become a part of the functional turn in the central region 108.

In certain examples, at least a portion of the coil 102 can be surrounded by a first cover 112. As shown in FIGS. 1C-1F, the first cover 112 can have a tubular shape and thus can also be referred to as a “tubular member.” In certain examples, the first cover 112 can cover an entire length of the coil 102. In certain examples, the first cover 112 covers only selected portion(s) of the coil 102. In some examples, as illustrated in FIGS. 1C-1D, at least a portion of the first cover 112 can be surrounded by the retention element 114. For example, in some examples, a distal end portion of the retention element 114 can extent axially beyond the distal end of the guard member 104 and be disposed at or adjacent a distal end of the coil 102, and a proximal end portion of the retention element 114 can extend axially beyond the proximal end of the guard member 104 and be disposed at or adjacent the ascending portion 110b of the coil 102. In some examples, as illustrated in FIGS. 1E-1F, at least a portion of the first cover 112 is not surrounded by the retention element 114.

In certain examples, the first cover 112 can be coated on and/or bonded on the coil 102. In certain examples, the first cover 112 can be a cushioned, padded-type layer protecting the coil. The first cover 112 can be constructed of various native and/or synthetic materials. In one particular example, the first cover can include expanded polytetrafluoroethylene (ePTFE). In certain examples, the first cover 112 is configured to be fixedly attached to the coil 102 (e.g., by means of textured surface resistance, suture, glue, thermal bonding, or any other means) so that relative axial movement between the first cover 112 and the coil 102 is restricted or prohibited.

As described herein, the guard member 104 can constitute a part of a cover assembly 120 for the docking device 100. In some examples, the cover assembly 120 can also include the first cover 112. In some examples, the cover assembly 120 can further include the retention element 114.

In some examples, as shown in FIGS. 1A-1B, when the docking device 100 is in the deployed configuration, the guard member 104 can be configured to cover a portion (e.g., the atrial portion 110a) of the stabilization turn 110 of the coil 102. In certain examples, the guard member 104 can be configured to cover at least a portion of the central region 108 of the coil 102, such as a portion of the proximal turn 108p. In certain examples, the guard member 104 can extend over the entirety of the coil 102.

As described herein, the guard member 104 can radially expand so as to help prevent and/or reduce paravalvular leakage. Specifically, the guard member 104 can be configured to radially expand such that an improved seal is formed closer to and/or against a prosthetic valve deployed within the docking device 100. In some examples, the guard member 104 can be configured to prevent and/or inhibit leakage at the location where the docking device 100 crosses between leaflets of the native valve (e.g., at the commissures of the native leaflets). For example, without the guard member 104, the docking device 100 may push the native leaflets apart at the point of crossing the native leaflets and allow for leakage at that point (e.g., along the docking device or to its sides). However, the guard member 104 can be configured to expand to cover and/or fill any opening at that point and inhibit leakage along the docking device 100.

In another example, when the docking device 100 is deployed at a native atrioventricular valve, the guard member 104 covers predominantly a portion of the stabilization turn 110 and/or a portion of the central region 108. For example, in one example, the guard member 104 can cover predominantly the atrial portion 110a of the stabilization turn 110 that is located distal to the ascending portion 110b (i.e., the guard member 104 does not extend into the ascending portion 110b when the docking device 100 is in the deployed configuration). In another example, the guard member 104 can cover not only the atrial portion 110a, but also extend over the ascending portion 110b of the stabilization turn 110. In various examples, the guard member 104 can help covering an atrial side of an atrioventricular valve to prevent and/or inhibit blood from leaking through the native leaflets, commissures, and/or around an outside of the prosthetic valve by blocking blood in the atrium from flowing in an atrial to ventricular direction (i.e., antegrade blood flow)-other than through the prosthetic valve.

In some examples, the guard member 104 can be positioned on a ventricular side of an atrioventricular valve to prevent and/or inhibit blood from leaking through the native leaflets, commissures, and/or around an outside of the prosthetic valve by blocking blood in the ventricle from flowing in a ventricular to atrial direction (i.e., retrograde blood flow).

The guard member 104 can include an expandable member 116 and a cover member 118 (also referred to as a “second cover” or an “outer cover”) surrounding an outer surface of the expandable member 116. In certain examples, the expandable member 116 surrounds at least a portion of the first cover 112. In certain examples, the first cover 112 can extend (completely or partially) through the expandable member 116.

The expandable member 116 can extend radially outwardly from the coil 102 (and the first cover 112) and is movable between a radially compressed (and axially elongated) state and a radially expanded (and axially foreshortened) state. That is, the expandable member 116 can axially foreshorten when it moves from the radially compressed state to the radially expanded state and can axially elongate when it moves from the radially expanded state to the radially compressed state.

In certain examples, the expandable member 116 can include a braided structure, such as a braided wire mesh or lattice. In certain examples, the expandable member 116 can include a shape memory material that is shape set and/or pre-configured to expand to a particular shape and/or size when unconstrained (e.g., when deployed at a native valve location). For example, the expandable member 116 can have a braided structure containing a metal alloy with shape memory properties, such as Nitinol. In another example, the expandable member 116 can include a foam structure. For example, the expandable member can include an expandable memory foam which can expand to a specific shape or specific pre-set shape upon removal of a crimping pressure (e.g., removal of the docking device 100 from the delivery sheath) prior to delivery of the docking device.

As described herein, the second cover 118 can be configured to be so elastic that when the expandable member 116 moves from the radially compressed (and axially elongated) state to the radially expanded (and axially foreshortened) state, the second cover 118 can also radially expand and axially foreshorten together with the expandable member 116. In other words, the guard member 104, as a whole, can move from a radially compressed (and axially elongated) state to a radially expanded (and axially foreshortened) state.

In certain examples, the second cover 118 can be configured to be atraumatic to native tissue and/or promote tissue ingrowth into the second cover 118. For example, the second cover 118 can have pores to encourage tissue ingrowth. In another example, the second cover 118 can be impregnated with growth factors to stimulate or promote tissue ingrowth, such as transforming growth factor alpha (TGF-alpha), transforming growth factor beta (TGF-beta), basic fibroblast growth factor (bFGF), vascular epithelial growth factor (VEGF), and combinations thereof. The second cover 118 can be constructed of any suitable material, including foam, cloth, fabric, and/or polymer, which is flexible to allow for compression and expansion of the second cover 118. In one example, the second cover 118 can include a fabric layer constructed from a thermoplastic polymer material, such as polyethylene terephthalate (PET).

In some examples, a distal end portion 104d of the guard member 104 (including a distal end portion of the expandable member 116 and a distal end portion of the second cover 118) can be fixedly coupled to the coil 102 (e.g., via a distal suture), and a proximal end portion 104p of the guard member 104 (including a proximal end portion of the expandable member 116 and a proximal end portion of the second cover 118) can be axially movable relative to the coil 102. Further, the proximal end portion of the expandable member 116 can be fixedly coupled to the proximal end portion of the second cover 118 (e.g., via a proximal suture).

When the docking device 100 is retained within the delivery sheath in the substantially straight configuration, the expandable member 116 can be radially compressed by the delivery sheath and remains in the radially compressed (and axially elongated) state. The radially compressed (and axially elongated) expandable member 116 can contact the retention element 114 (see, e.g., FIG. 1C) or the first cover 112 (see, e.g., FIG. 1E) so that no gap or cavity exists between the retention element 114 and the expandable member 116 or between the first cover 112 (and/or the coil 102) and the expandable member 116.

After the docking device 100 is removed from the delivery sheath and changes to the deployed configuration, the expandable member 116 can radially expand (and axially foreshorten) so that a gap or cavity 111 can be created between the retention element 114 and the expandable member 116 (see, e.g., FIG. 1D) and/or between the first cover 112 and the expandable member 116 (see, e.g., FIG. 1F).

Because the distal end portion 104d of the guard member 104 is fixedly coupled to the coil 102 and the proximal end portion 104p of the guard member 104 can be axially moveable relative to the coil 102, the proximal end portion 104p of the guard member 104 can slide axially over the first cover 112 and toward the distal end 102d of the coil 102 when expandable member 116 moves from the radially compressed state to the radially expanded state. As a result, the proximal end portion 104p of the guard member 104 can be disposed closer to the proximal end 102p of the coil 102 when the expandable member 116 is in the radially compressed state than in the radially expanded state.

In certain examples, the second cover 118 can be configured to engage with the prosthetic valve deployed within the docking device 100 so as to form a seal and reduce paravalvular leakage between the prosthetic valve and the docking device 100 when the expandable member 116 is in the radially expanded state. The second cover 118 can also be configured to engage with the native tissue (e.g., the native annulus and/or native leaflets) to reduce PVL between the docking device and/or the prosthetic valve and the native tissue.

In certain examples, when the expandable member 116 is in the radially expanded state, the proximal end portion 104p of the guard member 104 can have a tapered shape as shown in FIGS. 1A-1B, such that the diameter of the proximal end portion 104p gradually increases from a proximal terminal end of the guard member 104 to a distally located body portion of the guard member 104. This can, for example, help to facilitate loading the docking device into a delivery sheath of the delivery apparatus and/or retrieval and/or repositioning of the docking device into the delivery apparatus during an implantation procedure. In addition, due to its small diameter, the proximal terminal end of the guard member 104 can interact with the retention element 114 so that the retention element 114 can increase friction and reduce or prevent axial movement of the proximal end portion 104p of the guard member 104 relative to the coil 102.

In certain examples, the docking device 100 can include at least one radiopaque marker configured to provide visual indication about the location and/or the amount of radial expansion of the docking device 100 (e.g., when a prosthetic valve is subsequently deployed in the docking device 100) under fluoroscopy. In one example, one or more radiopaque markers can be placed on the coil 102. In another example, one or more radiopaque markers can be placed on the first cover 112, the expandable member 116, and/or the second cover 118. As noted above, one or more radiopaque markers (e.g., 113 and/or 115) can be placed on the proximal end portion 114p of the retention element 114.

Further details of the docking device and its variants, including various examples of the coil, the first cover, the second cover, the expandable member, and other components of the docking device, are described in PCT Patent Application Publication No. WO/2020/247907, the entirety of which is incorporated by reference herein.

### Exemplary Prosthetic Valves

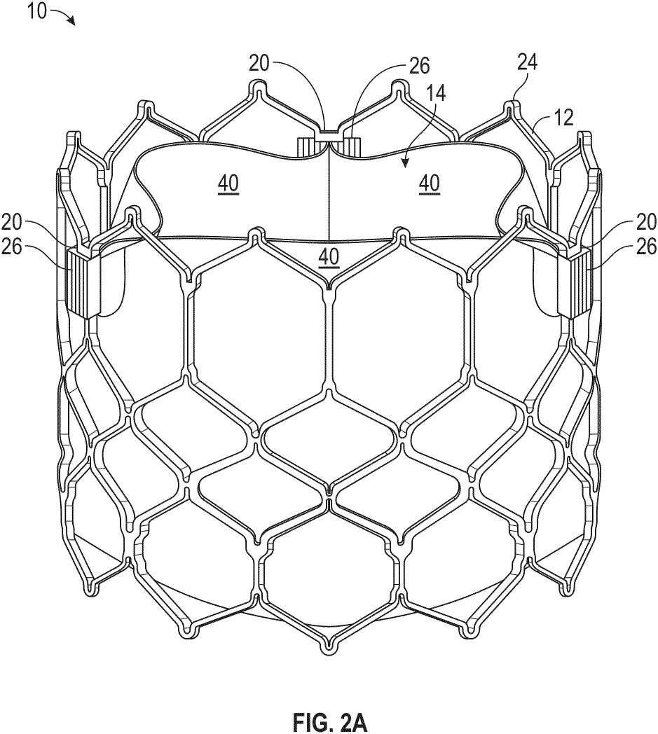

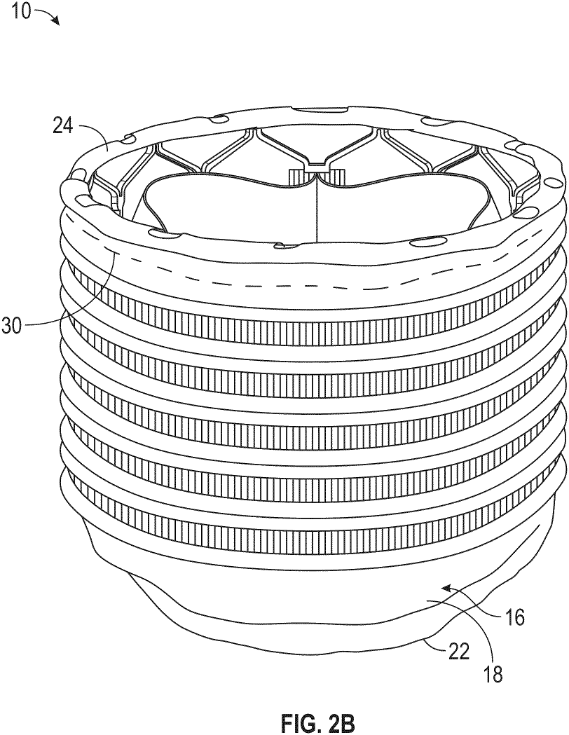

FIGS. 2A-2B show a prosthetic valve 10, according to one example. The prosthetic valve 10 can be adapted to be implanted, with or without a docking device, in a native valve annulus, such as the native mitral valve annulus, native aortic annulus, native pulmonary valve annulus, etc. The prosthetic valve 10 can include a stent, or frame, 12, a valvular structure 14, and a valve cover 16 (the valve cover 16 is removed in FIG. 2A to show the frame structure).

The valvular structure 14 can include three leaflets 40, collectively forming a leaflet structure (although a greater or fewer number of leaflets can be used), which can be arranged to collapse in a tricuspid arrangement. The leaflets 40 are configured to permit the flow of blood from an inflow end 22 to an outflow end 24 of the prosthetic valve 10 and block the flow of blood from the outflow end 24 to the inflow end 22 of the prosthetic valve 10. The leaflets 40 can be secured to one another at their adjacent sides to form commissures 26 of the leaflet structure. The lower edge of valvular structure 14 desirably has an undulating, curved scalloped shape. By forming the leaflets 40 with this scalloped geometry, stresses on the leaflets 40 can be reduced, which in turn can improve durability of the prosthetic valve 10. Moreover, by virtue of the scalloped shape, folds and ripples at the belly of each leaflet 40 (the central region of each leaflet), which can cause early calcification in those areas, can be eliminated or at least minimized. The scalloped geometry can also reduce the amount of tissue material used to form leaflet structure, thereby allowing a smaller, more even crimped profile at the inflow end of the prosthetic valve 10. The leaflets 40 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

The frame 12 can be formed with a plurality of circumferentially spaced slots, or commissure windows 20 (three in the illustrated example) that are adapted to mount the commissures 26 of the valvular structure 14 to the frame. The frame 12 can be made of any of various suitable plastically expandable materials (e.g., stainless steel, etc.) or self-expanding materials (e.g., Nitinol) as known in the art. When constructed of a plastically expandable material, the frame 12 (and thus the prosthetic valve 10) can be crimped to a radially compressed state on a delivery apparatus and then expanded inside a patient by an inflatable balloon or equivalent expansion mechanism. When constructed of a self-expandable material, the frame 12 (and thus the prosthetic valve 10) can be crimped to a radially compressed state and restrained in the compressed state by insertion into a delivery sheath or equivalent mechanism of a delivery apparatus. Once inside the body, the prosthetic valve 10 can be advanced from the delivery sheath, which allows the prosthetic valve 10 to expand to its functional size.

Suitable plastically expandable materials that can be used to form the frame 12 include, without limitation, stainless steel, a nickel-based alloy (e.g., a cobalt-chromium or a nickel-cobalt-chromium alloy), polymers, or combinations thereof. In particular examples, frame 12 can be made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight. It has been found that the use of MP35N to form the frame 12 can provide superior structural results over stainless steel. In particular, when MP35N is used as the frame material, less material is needed to achieve the same or better performance in radial and crush force resistance, fatigue resistances, and corrosion resistance. Moreover, since less material is required, the crimped profile of the frame can be reduced, thereby providing a lower profile valve assembly for percutaneous delivery to the treatment location in the body.

As shown in FIG. 2B, the valve cover 16 can include an outer portion 18 which can cover an entire outer surface of the frame 12. In certain examples, as shown in FIG. 3A, the valve cover 16 can also include an inner portion 28 which can cover an entire inner surface of the frame 12, or alternatively, cover only selected portions of the inner surface of the frame 12. The valve cover 16 can be affixed to the frame 12 by a variety of means, such as via sutures 30.

As described herein, the valve cover 16 can be configured to prevent paravalvular leakage between the prosthetic valve 10 and the native valve, to protect the native anatomy, to promote tissue ingrowth, among some other purposes. For mitral valve replacement, due to the general D-shape of the mitral valve and relatively large annulus compared to the aortic valve, the valve cover 16 can act as a seal around the prosthetic valve 10 (e.g., when the prosthetic valve 10 is sized to be smaller than the annulus) and allows for smooth coaptation of the native leaflets against the prosthetic valve 10.

In various examples, the valve cover 16 can include a material that can be crimped for transcatheter delivery of the prosthetic valve 10 and is expandable to prevent paravalvular leakage around the prosthetic valve 10. Examples of possible materials include foam, cloth, fabric, one or more synthetic polymers (e.g., polyethylene terephthalate (PET), polytetrafluoroethylene (PTFE), ePTFE, etc.), organic tissues (e.g., bovine pericardium, porcine pericardium, equine pericardium, etc.), and/or an encapsulated material (e.g., an encapsulated hydrogel).

In certain examples, the valve cover 16 can be made of a woven cloth or fabric possessing a plurality of floated yarn sections 32 (e.g., protruding or puffing sections, also referred to as “floats” hereinafter). Details of exemplary covered valves with a plurality of floats 32 are further described in U.S. Pat. Publication Nos. US2019/0374337, US2019/0192296, and US2019/0046314, the disclosures of which are incorporated herein in their entireties for all purposes. In certain examples, the float yarn sections 32 can be separated by one or more horizontal bands 34. In some examples, the horizontal bands 34 can be constructed via a leno weave, which can improve the strength of the woven structure. In some examples of the woven cloth, vertical fibers (e.g., running along the longitudinal axis of the prosthetic valve 10) can include a yarn or other fiber possessing a high level of expansion, such as a texturized weft yarn, while horizontal fibers (e.g., running circumferentially around the prosthetic valve 10) in a leno weave can include a low expansion yarn or fiber.

In some examples, the valve cover 16 can include a woven cloth resembling a greige fabric when assembled and under tension (e.g., when stretched longitudinally on a compressed valve prior to delivery of a prosthetic valve 10). When the prosthetic valve 10 is deployed and expanded, tension on floats 32 is relaxed allowing expansion of the floats 32. In some examples, the valve cover 16 can be heat set to allow floats 32 to return to an enlarged, or puffed, space-filling form. In some examples, the number and sizes of floats 32 can be optimized to provide a level of expansion to prevent paravalvular leakage across the mitral plane (e.g., to have a higher level of expansion thickness) and/or a lower crimp profile (e.g., for delivery of the prosthetic valve). Additionally, the horizontal bands 34 can be optimized to allow for attachment of the valve cover 16 to the frame 12 based on the specific size or position of struts or other structural elements on the prosthetic valve 10.

Further details of the prosthetic valve 10 and its components are described, for example, in U.S. Pat. Nos. 9,393,110 and 9,339,384, which are incorporated by reference herein. Additional examples of the valve cover are described in PCT Patent Application Publication No. WO/2020/247907.

As described above and illustrated in FIGS. 3A-3B, the prosthetic valve 10 can be radially expanded and securely anchored within the docking device 100.

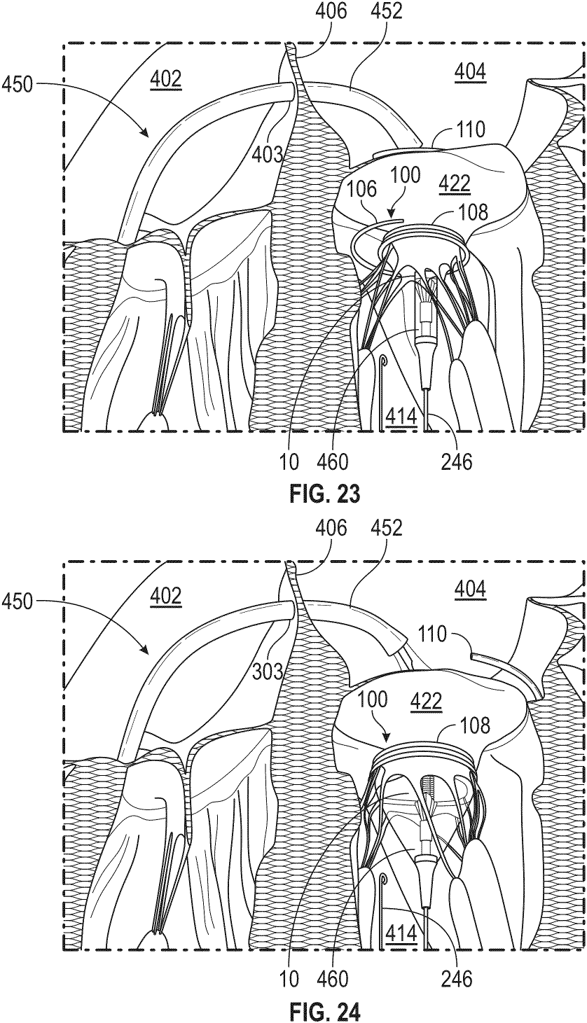

In certain examples, and as described further below in reference to FIGS. 23-24, the coil 102 of the docking device 100 in the deployed configuration can be movable between a first radially expanded configuration before the prosthetic valve 10 is radially expanded within the coil 102 and a second radially expanded configuration after the prosthetic valve 10 is radially expanded within the coil 102. In the example depicted in FIGS. 3A-3B, the coil 102 is in the second radially expanded configuration since the prosthetic valve 10 is shown in the radially expanded state.

As described herein, at least a portion of the coil 102, such as the central region 108, can have a larger diameter in the second radially expanded configuration than in the first radially expanded configuration. As the central region 108 increases in diameter when the coil 102 moves from the first radially expanded configuration to the second radially expanded configuration, a distance between the proximal end 102p and the distal end 102d of the coil 102 can be foreshortened correspondingly.

### Exemplary Cover Assembly

As described above, the docking device 100 can have a cover assembly 120 including the first cover 112 and the guard member 104, and in some instances the retention element 114. The guard member 104 can further include the expandable member 116 and the second cover 118. As described herein, the second cover 118 can be fixedly coupled to the expandable member 116 so that the second cover 118 can radially expand and axially foreshorten together with the expandable member 116.

In one example, the cover assembly 120 can be assembled by fixedly attaching the distal end portion 104d of the guard member 104 to the coil 102 (and the first cover 112 surrounding the coil 102) while leaving the proximal end portion 104p of the guard member 104 unattached to the coil 102 (and the first cover 112 surrounding the coil 102). Thus, the proximal end portion 104p can be axially movable relative to the coil 102 and the first cover 112. As a result, when the coil 102 moves from the delivery configuration to the deployed configuration (e.g., during the initial deployment of the docking device 100), the proximal end portion 104p of the guard member 104 can slide distally over the coil 102 to cause the guard member 104 to contract axially (i.e., with decrease of axial length) while it expands radially (i.e., with increase in diameter).

On the other hand, the retention element 114, by applying a friction force, can limit the extent of distal movement of the proximal end portion 104p. For example, if the proximal end portion 104p of a fully expanded guard member 104 (i.e., expanding to its largest diameter) can slide distally over the coil 102 to a first location in the absence of retention element 114, then the presence of the retention element 114 can cause the proximal end portion 104p to slide distally over the coil 102 to a second location that is proximal to the first location. In other words, the retention element 114 can prevent the guard member 104 to expand to its largest diameter and/or contract to its shortest axial length.

The guard member 104 can be coupled to the coil 102 and/or first cover 112 in various ways such as adhesive, fasteners, welding, and/or other means for coupling. For example, in some examples, attaching the second cover 118 to the expandable member 116 or attaching the distal end portion 104d of the guard member to the coil 102 and the first cover 112 can be achieved by using one or more sutures. In one particular example, a distal end portion of the second cover 118 and a distal end portion of the expandable member 116 can be fixedly coupled to the coil 102 via a distal suture. In addition, a proximal end portion of the expandable member 116 can be fixedly coupled to a proximal end portion of the second cover 118 via a proximal suture. An example method assembling the cover assembly is described in U.S. Provisional Application Ser. No. 63/252,524, the entirety of which is incorporated by reference herein.

### Exemplary Delivery Apparatus

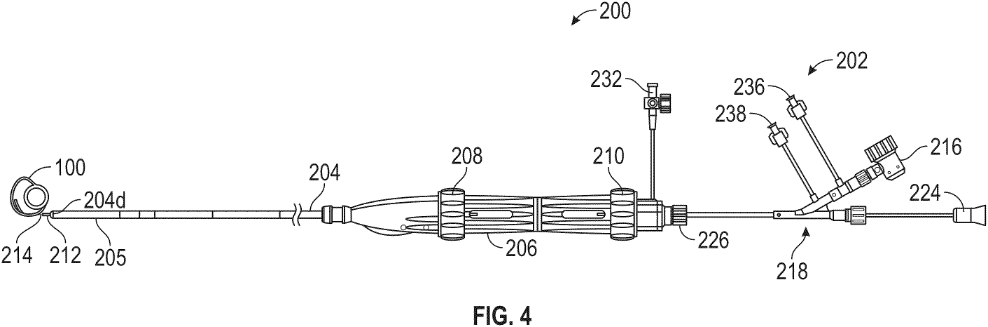

FIG. 4 shows a delivery apparatus 200 configured to implant a docking device, such as the docking device 100 described above or other docking devices, to a target implantation site in a patient, according to one example. Thus, the delivery apparatus 200 can also be referred to as a “dock delivery catheter” or “dock delivery system.”

As shown, the delivery apparatus 200 can include a handle assembly 202 and a delivery sheath 204 (also referred to as the “delivery shaft” or “outer shaft” or “outer sheath”) extending distally from the handle assembly 202. The handle assembly 202 can include a handle 206 including one or more knobs, buttons, wheels, and/or other means for controlling and/or actuating one or more components of the delivery apparatus 200. For example, in some examples, as shown in FIG. 4, the handle 206 can include knobs 208 and 210 which can be configured to steer or control flexing of the delivery apparatus 200 such as the delivery sheath 204 and/or the sleeve shaft 220 described below.

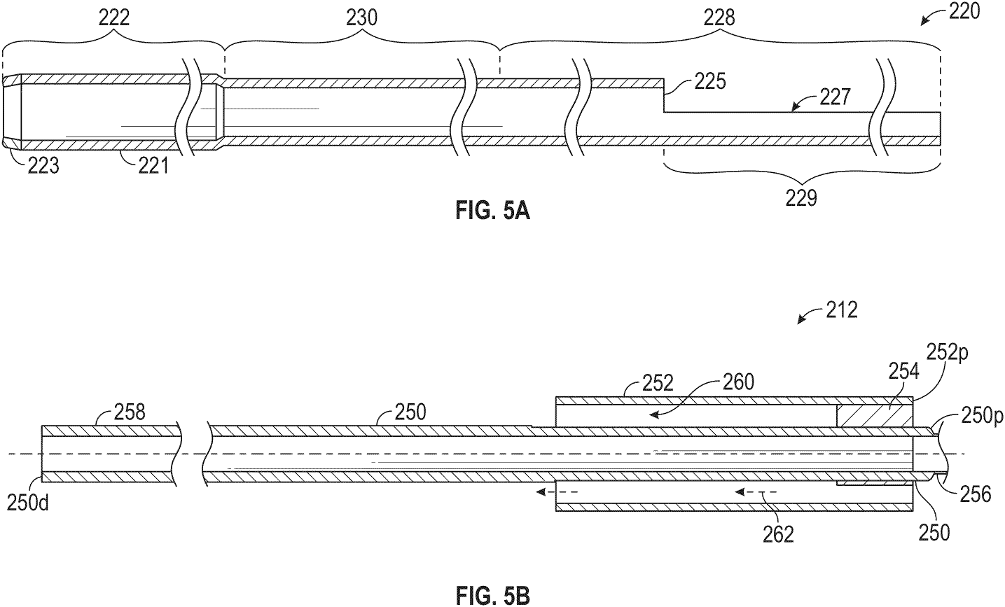

In certain examples, the delivery apparatus 200 can also include a pusher shaft 212 (see, e.g., FIG. 5B) and a sleeve shaft 220 (see, e.g., FIG. 5A), both of which can extend through an inner lumen of the delivery sheath 204 and have respective proximal end portions extending into the handle assembly 202.

As described below, a distal end portion (also referred to as “distal section”) of the sleeve shaft 220 can include a lubricous dock sleeve 222 configured to cover (e.g., surround) the docking device 100. For example, the docking device 100 can be retained inside the dock sleeve 222, which is further retained by a distal end portion 205 of the delivery sheath 204, when navigating through a patient’s vasculature. As noted above, the docking device 100 retained within the delivery sheath 204 can remain in the delivery configuration.

Additionally, the distal end portion 205 of the delivery sheath 204 can be configured to be steerable. In one example, by rotating a knob (e.g., 208 or 210) on the handle 206, a curvature of the distal end portion 205 can be adjusted so that the distal end portion 205 of the delivery sheath 204 can be oriented in a desired angle. For example, as shown in FIG. 14 and described below, to implant the docking device 100 at the native mitral valve location, the distal end portion 205 of the delivery sheath 204 can be steered in the left atrium so that the dock sleeve 222 and the docking device 100 retained therein can extend through the native mitral valve annulus at a location adjacent the posteromedial commissure.

In certain examples, the pusher shaft 212 and the sleeve shaft 220 can be coaxial with one another, at least within the delivery sheath 204. In addition, the delivery sheath 204 can be configured to be axially movable relative to the sleeve shaft 220 and the pusher shaft 212. As described further below, a distal end of the pusher shaft 212 can be inserted into a lumen of the sleeve shaft 220 and press against the proximal end (e.g., 102d) of the docking device 100 retained inside the dock sleeve 222.

After reaching a target implantation site, the docking device 100 can be deployed from the delivery sheath 204 by manipulating the pusher shaft 212 and sleeve shaft 220 using a hub assembly 218, as described further below. For example, by pushing the pusher shaft 212 in the distal direction while holding the delivery sheath 204 in place or retracting the delivery sheath 204 in the proximal direction while holding the pusher shaft 212 in place, or pushing the pusher shaft 212 in the distal direction while simultaneously retracting the delivery sheath 204 in the proximal direction, the docking device 100 can be pushed out of a distal end 204d of the delivery sheath 204, thus changing from the delivery configuration to the deployed configuration.

In certain examples, the pusher shaft 212 and the sleeve shaft 220 can be actuated independently of each other. In certain examples, when deploying the docking device 100 from the delivery sheath 204, the pusher shaft 212 and the sleeve shaft 220 can be configured to move together, in the axial direction, with the docking device 100. For example, actuation of the pusher shaft 212, to push against the docking device 100 and move it out of the delivery sheath 204 can also cause the sleeve shaft 220 to move along with the pusher shaft 212 and the docking device 100. As such, the docking device 100 can remain being covered by the dock sleeve 222 of the sleeve shaft 220 during the procedure of pushing the docking device 100 into position at the target implantation site via the pusher shaft 212. Thus, when the docking device 100 is initially deployed at the target implantation site, the lubricous dock sleeve 222 can facilitate the covered docking device 100 to encircle the native anatomy.

During delivery, the docking device 100 can be coupled to the delivery apparatus 200 via a release suture 214 (or other retrieval line comprising a string, yarn, or other material that can be configured to be tied around the docking device 100 and cut for removal) that extends through the pusher shaft 212. In one specific example, the release suture 214 can extend through the delivery apparatus 200, e.g., through an inner lumen of the pusher shaft 212, to a suture lock assembly 216 of the delivery apparatus 200.

The handle assembly 202 can further include a hub assembly 218 to which the suture lock assembly 216 and a sleeve handle 224 are attached. The hub assembly 218 can be configured to independently control the pusher shaft 212 and the sleeve shaft 220 while the sleeve handle 224 can control an axial position of the sleeve shaft 220 relative to the pusher shaft 212. In this way, operation of the various components of the handle assembly 202 can actuate and control operation of the components arranged within the delivery sheath 204. In some examples, the hub assembly 218 can be coupled to the handle 206 via a connector 226.

The handle assembly 202 can further include one or more flushing ports (e.g., three flushing ports 232, 236, 238 are shown in FIG. 4) to supply flush fluid to one or more lumens arranged within the delivery apparatus 200 (e.g., annular lumens arranged between coaxial components of the delivery apparatus 200), as described below.

Further details on delivery apparatus/catheters/systems (including various examples of the handle assembly) that are configured to deliver a docking device to a target implantation site can be found in U.S. Pat. Publication Nos. 2018/0318079 and 2018/0263764, which are all incorporated by reference herein in their entireties.

### Exemplary Sleeve Shaft

FIG. 5A shows a sleeve shaft 220, according to one example. In some examples, the sleeve shaft 220 can have a lubricous distal section 222 (also referred to as the “dock sleeve” herein) configured to cover a docking device (e.g., 100) during deployment, a proximal section 228 used to manipulate or actuate position of the distal section 222, and a middle section 230 connecting the distal section 222 and the proximal section 228.

In some examples, the dock sleeve 222 can be configured to be flexible, have a lower durometer than the remainder of the sleeve shaft 220, and have a hydrophilic coating, which can act as a lubricous surface to improve the ease of encircling the native anatomy and reduce risk of damage to the native tissue. In some examples, the dock sleeve 222 can form a tubular structure which has an inner diameter sufficient to surround the docking device 100 and an outer diameter that is small enough to be retained within and axially movable within the delivery sheath 204. In some examples, the outer diameter of the dock sleeve 222 can be slightly larger than the outer diameter of the middle section 230. In some examples, the length of the dock sleeve 222 is sufficient to cover or longer than the full length of the docking device 100 when it is retained inside the dock sleeve 222.

The dock sleeve 222 can have a body portion 221 and a tip portion 223 located at a distal end of the body portion 221. In some examples, the tip portion 223 can extend about 1-4 mm (e.g., about 2 mm) distally from the distal end of the body portion 221. In some examples, the tip portion 223 can taper radially inwardly such that it has a smaller diameter than the body portion 221. In some examples, during delivery, the tip portion 223 can extend past the distal end (e.g., 102d) of the docking device, thereby providing the dock sleeve 222 with a more atraumatic tip that can bend, squeeze, deform, or the like, as it is navigated around the native architecture of the implantation site for the docking device. Examples of the dock sleeve, including alternative designs of the tip portion, are described further below.

In some examples, the middle section 230 of the sleeve shaft 220 can be configured to provide a sufficient column strength so as to push the dock sleeve 222 (with the docking device 100) out of a distal end 204d of the delivery sheath 204, and/or retract the dock sleeve 222 after the docking device 100 is deployed at the target implantation site. The middle section 230 can also be configured to have an enough flexibility so as to facilitate navigating the anatomy of a patient from the point of insertion of the delivery apparatus 200 to the heart. In certain examples, the dock sleeve 222 and the middle section 230 can be formed as a single, continuous unit with varying properties (e.g., dimensions, polymers, braids, etc.) along the length of the singular unit.

In some examples, a proximal portion of the proximal section 228 can be arranged in the handle assembly 202. The proximal section 228 of the sleeve shaft 220 can be configured to be more rigid and provide column strength to actuate the position of the dock sleeve 222 by pushing the middle section 230 and dock sleeve 222 with the docking device 100 and retracting the dock sleeve 222 after the docking device 100 is deployed at the target implantation site.

In some examples, the proximal portion of the proximal section 228 can include a cut portion 229 which has a cross-section (in a plane normal to a central longitudinal axis of the sleeve shaft 220) that is not a complete circle (e.g., is open and does not form a closed tube). An end surface 225 can be formed between the cut portion 229 and the remainder of the proximal section 228. The end surface 225 can be configured normal to a central longitudinal axis of the sleeve shaft 220 and can be configured to come into contact with a stop element (e.g., plug 254) of the pusher shaft 212, as explained further below.

The cut portion 229 can extend into the hub assembly 218 of the handle assembly 202. As described below, a proximal extension 256 of the pusher shaft 212 can extend along an inner surface of the cut portion 229. The cut (e.g., open) profile of the cut portion 229 can allow the proximal extension 256 of the pusher shaft 212 to extend out of a void space 227 formed in the cut portion 229 and branch off, at an angle relative to the cut portion 229, into the suture lock assembly 216 of the hub assembly 218 (see, e.g., FIG. 4). As such, the pusher shaft 212 and sleeve shaft 220 can be operated in parallel with one another and an overall length of the delivery apparatus 200 in which the sleeve shaft 220 and pusher shaft 1900 are incorporated can be maintained similar to or only minimally longer than a delivery system that does not incorporate the sleeve shaft 220.

Additional examples of the sleeve shaft are described further in PCT Patent Application Publication No. WO/2020/247907.

### Exemplary Pusher Shaft

FIG. 5B shows a pusher shaft 212, according to one example. As shown, the pusher shaft 212 can include a main tube 250, a shell 252 surrounding a proximal end portion of the main tube 250, a plug 254 connecting the main tube 250 to the shell 252, and a proximal extension 256 extending from a proximal end of the main tube 250.

The main tube 250 can be configured for advancing and retracting a docking device (such as one of the docking devices described herein) and housing the release suture (e.g., 214) that secures the docking device to the pusher shaft 212. The main tube 250 can extend from the distal end 204d of the delivery sheath 204 into the handle assembly 202 of the delivery apparatus 200. For example, in certain examples, a proximal end portion of the pusher shaft 212, which includes an interface between the main tube 250, the shell 252, the plug 254, and the proximal extension 256, can be arranged within or proximate to the hub assembly 218 of the handle assembly 202. Thus, the main tube 250 can be an elongate tube that extends along a majority of the delivery apparatus 200.

The main tube 250 can be a relatively rigid tube that provides column strength for actuating deployment of a docking device. In some examples, the main tube 250 can be a hypo tube. In some examples, the main tube 250 can comprise a biocompatible metal, such as stainless steel. The main tube 250 can have a distal end 250d configured to interface with a docking device and a proximal end 250p, where the proximal extension 256 is attached. In some examples, a distal section 258 of the main tube 250 can be relatively more flexible (e.g., via one or more cuts into an outer surface of the main tube and/or having a durometer material) than the remaining part of the main tube 250. Thus, the distal section 258 can flex and/or bend along with the delivery sheath 204 of the delivery apparatus 200, as it is navigated through a vasculature of a patient, to the target implantation site.

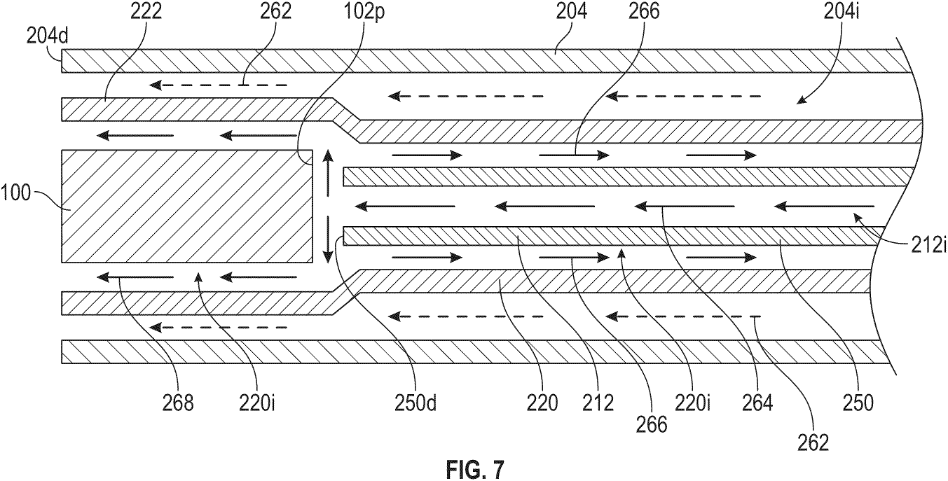

In some examples, the shell 252 can be configured to lock the main tube 250 and provide a hemostatic seal on the pusher shaft 212 without interfering with movement of the sleeve shaft 220. As shown in FIG. 5B, an inner diameter of the shell 252 can be larger than an outer diameter of the main tube 250, thereby forming an annular cavity 260 between the main tube 250 and the shell 252. As such, the proximal section 228 of the sleeve shaft 220 can slide within the annular cavity 260, as described further below. Further, flush fluid provided to a lumen on an exterior of the proximal extension 256, in the hub assembly 218, can flow through the annular cavity 260 and exit at a distal end of the shell 252 (as shown by arrows 262) to enter a lumen between the sleeve shaft 220 and delivery sheath 204 of the delivery apparatus, as discussed further below with reference to FIG. 7.

The plug 254 can be configured to be arranged within the annular cavity 260, at a proximal end 252p of the shell 252. In some examples, the plug 254 can be configured to “plug” or fill a portion of the annular cavity 260 located at the proximal end 252p of the shell 252, while leaving the remaining portion of the annular cavity 260 open to receive the cut portion 229 of the sleeve shaft 220 therein. In some examples, the shell 252 and the plug 254 can be fixedly coupled to the main tube 250 (e.g., via welding) to allow the cut portion 229 of the sleeve shaft 220 to slide between the main tube 250 and the shell 252. As described below, the plug 254 can also act as a stop for the sleeve shaft 220.

As noted above, the proximal extension 256 can extend from the proximal end 250p of the main tube 250 and the shell 252. The proximal extension 256 can provide the pusher shaft 212 with certain flexibility such that it may be routed from the inside of the sleeve shaft 220 (e.g., the cut portion 229) to the outside of the sleeve shaft 220, thereby allowing the pusher shaft 212 and the sleeve shaft 220 to be actuated in parallel and reducing an overall length of the delivery apparatus. In certain examples, the proximal extension 256 can be made of a flexible polymer.

Additional examples of the pusher shaft are described further in PCT Patent Application Publication No. WO/2020/247907.

### Exemplary Sleeve Shaft and Pusher Shaft Assembly

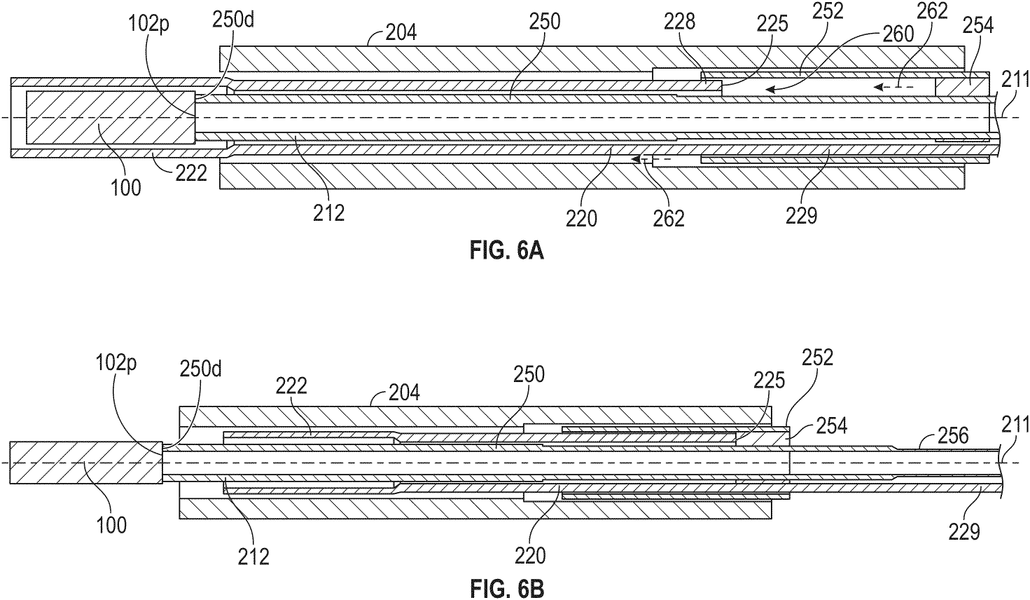

FIGS. 6A-6B illustrate example arrangements of the pusher shaft 212 and sleeve shaft 220 in the delivery sheath 204 of the delivery apparatus 200, before and after deployment of a docking device such as 100. As shown, the main tube 250 of the pusher shaft 212 can extend through a lumen of the sleeve shaft 220, which can extend through a lumen of the delivery sheath 204. The pusher shaft 212 and the sleeve shaft 220 can share a central longitudinal axis 211 of the delivery sheath 204.

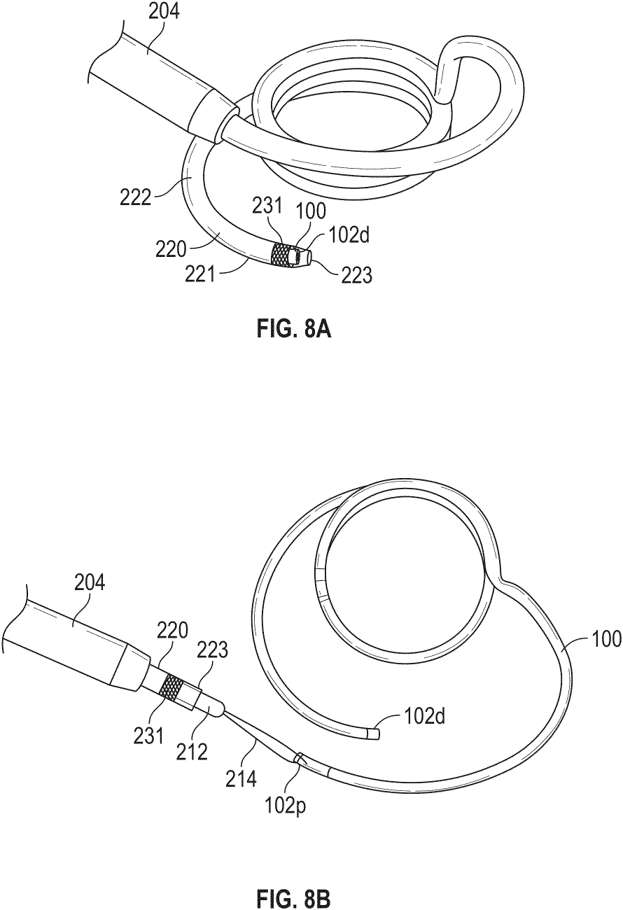

FIG. 7 shows various lumens configured to receive flush fluid during a delivery and implantation procedure can be formed between the docking device 100, the pusher shaft 212, the sleeve shaft 220, and the delivery sheath 204. Additionally, FIG. 8A shows a first configuration where the docking device 100 has been deployed from the delivery sheath 204 while still being covered by the dock sleeve 222 of the sleeve shaft 220 (the guard member 104 of the docking device is not shown for clarity purposes). The dock sleeve 222 in the first configuration is also referred to be in a “covered state.” FIG. 8B shows a second configuration where the docking device 100 is uncovered by the dock sleeve 222 after the sleeve shaft 220 has been retracted back into the delivery sheath 204 (the guard member 104 of the docking device is not shown). The dock sleeve 222 in the second configuration is also referred to be in an “uncovered state.”

Specifically, FIG. 6A illustrates a first configuration of the pusher shaft 212 and sleeve shaft 220 assembly, pre-deployment or during deployment of the docking device 100, according to one example. As shown, the dock sleeve 222 can be configured to cover the docking device 100 while the end surface 225 of the sleeve shaft 220 is positioned away from the plug 254. In addition, the distal end 250d of the pusher shaft 212 can extend into the dock sleeve 222 and come into contact with the proximal end 102p of the docking device 100.

During deploying the docking device 100 from the delivery sheath 204, the pusher shaft 212 and the sleeve shaft 220 can be configured to move together, in the axial direction, with the docking device 100. For example, actuation of the pusher shaft 212, to push against the docking device 100 and move it out of the delivery sheath 204 can also cause the sleeve shaft 220 to move along with the pusher shaft 212 and the docking device 100. As such, the docking device 100 can remain being covered by the dock sleeve 222 of the sleeve shaft 220 during the procedure of pushing the docking device 100 into position at the target implantation site via the pusher shaft 212, as illustrated in FIG. 8A.

Additionally, as shown in FIG. 8A, during delivery and implantation of the covered docking device 100 at the target implantation site, the tip portion 223 of the sleeve shaft 220 can extend distal to the distal end 102d of the docking device 100, thereby providing the dock sleeve 222 with a more atraumatic tip. In some examples, a radiopaque material (e.g., in the form of one or more marker bands 231, can be placed at the dock sleeve 222, e.g., at the intersection between the body portion 221 and the tip portion 223. In some examples, the distal end 102d of the docking device 100 can be arranged proximate to or just distal to the marker band 231 of the dock sleeve 222.

FIG. 6B illustrates a second configuration of the pusher shaft 212 and sleeve shaft 220 assembly, after deploying the docking device 100 from the delivery sheath 204 at the target implantation site and removing the dock sleeve 222 from the implanted docking device 100, according to one example. As shown, after implanting the docking device 100 at the target implantation site, in its desired position, the sleeve shaft 220 can be pulled off the docking device 100 and retracted back into delivery sheath 204 while holding the pusher shaft 212 steady so that its distal end 250d presses against the proximal end 102p of the docking device 100. Alternatively, the docking device 100 can be exposed by pushing the pusher shaft 212 in the distal direction while holding the sleeve shaft 220 steady. In some examples, as shown in FIG. 6B, the sleeve shaft 220 can be stopped from further retraction into the delivery apparatus upon the end surface 225 coming into contact with the plug 254.

FIG. 8B shows the sleeve shaft 220 removed from the docking device 100, leaving the docking device 100 uncovered by the dock sleeve 222. As shown, the tip portion 223 of the sleeve shaft 220 can be arranged proximal to (e.g., retracted past) the distal end of the pusher shaft 212 which can still be connected to the proximal end 102p of the docking device 100 via the release suture 214. As explained further below, after implanting the docking device 100 at the target implantation site and removing the dock sleeve 222 from covering the docking device 100, the docking device 100 can be disconnected from the delivery apparatus by cutting the release suture 214, e.g., by using the suture lock assembly 216 of the delivery apparatus 200.

As shown in FIG. 7, a first, pusher shaft lumen 212i can be formed within an interior of the pusher shaft 212 (e.g., within an interior of the main tube 250). The pusher shaft lumen 212i can receive a flush fluid from a first fluid source, which may be fluidly coupled to a portion of the handle assembly 202. The flush fluid flow 264 through the pusher shaft lumen 212i can travel along a length of the main tube 250 of the pusher shaft 212, to the distal end 250d of the main tube 250 of the pusher shaft 212. In some examples, the distal end 250d of the main tube 250 can be spaced away from the proximal end 102p of the docking device 100. Thus, at least a portion of the flush fluid flow 264 can flow into a distal portion of a second, sleeve shaft lumen 220i, which is arranged between an outer surface of the docking device 100 and an inner surface of the dock sleeve 222 of the sleeve shaft 220, as flush fluid flow 268. Further, in some examples, a portion of the flush fluid flow 264 can also flow back into a proximal portion of the sleeve shaft lumen 220i, which is arranged between an outer surface of the pusher shaft 212 and an inner surface of the sleeve shaft 220 that is proximal to the dock sleeve 222, as flush fluid flow 266. Thus, the same, first fluid source may provide flush fluid to the pusher shaft lumen 212i the sleeve shaft lumen 220i (including both the distal portion outside the dock sleeve 222 and the proximal portion that is proximal to the dock sleeve 222), via the pusher shaft lumen 212i.

FIG. 7 also shows a third, delivery sheath lumen 204i, which is arranged between an inner surface of the delivery sheath 204 and an outer surface of the sleeve shaft 220. The delivery sheath lumen 204i can receive a flush fluid from one or more second fluid sources, which may be fluidly coupled to a portion of the handle assembly 202, and which may result in flush fluid flow 262 flowing through the delivery sheath lumen 204i, to the distal end 204d of the delivery sheath 204.

Flushing the above-described lumens can be important to prevent thrombosis on and around the docking device 100 and other concentric parts of the delivery apparatus 200 during deployment of the docking device 100 from the delivery apparatus 200 and implantation of the docking device 100 at a target implantation site. In an example, as shown in FIG. 4, the first and/or the second fluid sources can be connected to one or more flushing ports (e.g., 232, 236, 238) arranged on and/or coupled to the handle assembly 202 of the delivery apparatus 200 to provide the flush fluid to the lumens described above.

Additional examples of the sleeve shaft and pusher shaft assembly are described further in PCT Patent Application Publication No. WO/2020/247907.

### Exemplary Dock Sleeve

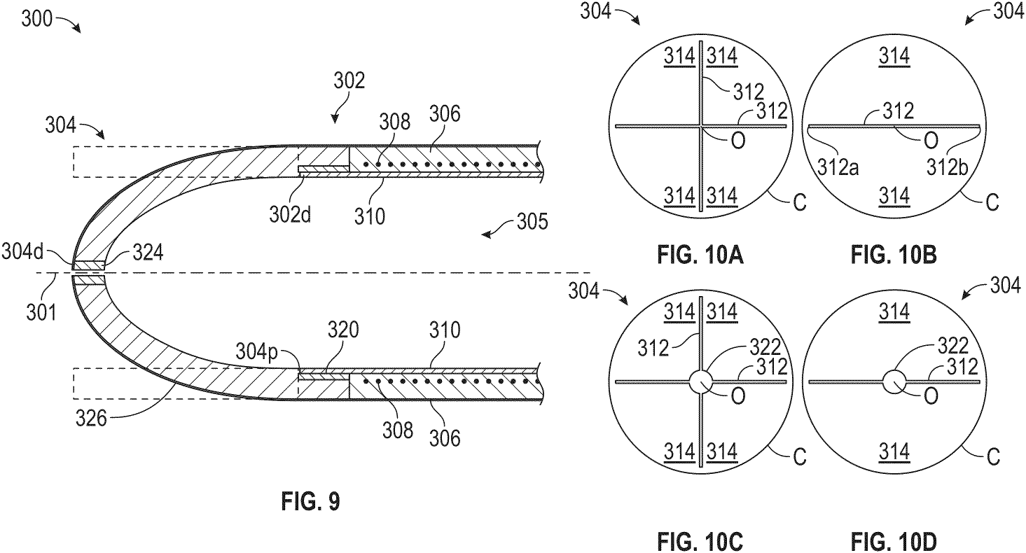

FIG. 9 shows a distal portion of a dock sleeve 300, according to another example. Similar to dock sleeve 222, the dock sleeve 300 can form a distal section of a sleeve shaft (e.g., 220) configured to cover a docking device (e.g., 100). As shown, the dock sleeve 300 has a body portion 302 and a tip portion 304 connected to a distal end 302d of the body portion 302.

The body portion 302 can have a generally tubular or cylindrical shape with a lumen 305 configured to receive at least a distal portion of the docking device (e.g., 100). Thus, when the distal end 302d of the body portion 302 axially aligns with the distal end (e.g., 102d) of the docking device (e.g., 100), at least the distal portion of the docking device (e.g., 100) can be covered by the body portion 302. In certain examples, the lumen 305 is configured to receive an entire length of the docking device in its delivery configuration.

The tip portion 304 can have a tapered shape extending distally relative to the body portion 302. As described further below, the tip portion 304 can be movable between a radially collapsed state and a radially expanded state. When the body portion 302 covers the distal portion of the docking device (e.g., 100), the tip portion 304 in the radially collapsed state can cover the distal end (e.g., 102d) of the docking device, and the tip portion 304 in the radially expanded state can allow the distal end (e.g., 102d) of the docking device to move distally relative to the distal end 302d of the body portion 302.

As shown, the body portion 302 of the dock sleeve 300 can, in some examples, include a plurality of layers and/or multiple components. In some examples, the body portion can comprise a main layer and a reinforcing element. In certain examples, the body portion 302 can comprise a flexible polymer jacket 306 reinforced by a braided layer or braid 308. In some examples, the polymer jacket 306 can extend axially along an entire length of the body portion 302. In some examples, the polymer jacket 306 can extend into the middle section (e.g., 230) of the sleeve shaft. In some examples, as illustrated in FIG. 9, the braid 308 does not extend into the distal tip portion 304 of the dock sleeve 300.

The flexible polymer jacket 306 can be selected from a variety of elastomeric materials, while the braid 308 can be configured to be supportive and flexible. In some examples, the braid 308 can be constructed of metals, such as nitinol or stainless steel. In certain examples, the flexible polymer can be a polyether-amide block copolymer or a blend of two or more polyether-amide block copolymers. In certain examples, the flexible polymer can be one of or a blend of two or more of PEBAX® grades 2533, 3533, 4033, 4533, and 5513 (Arkema S.A., France) and VESTAMID® grade E40 (Evonik Industries AG, Germany). In some examples, the flexible polymer can be PEBAX® 2533. In some examples, the flexible polymer can include other low durometer thermoplastic elastomers, such as chronoprene, santoprene, tecothane, to name a few.

The tip portion 304 of the dock sleeve 300 can be constructed of a flexible polymeric material. In some examples, the tip portion 304 can be constructed of the same material as the polymer jacket 306, and the tip portion 304 and the body portion 302 can be formed as a unitary piece. In some examples, the tip portion 304 can be constructed of a different polymeric material than the polymer jacket 306. For example, the tip portion 304 can be constructed of a polymeric material that has a lower flexural modulus than the material forming the polymer jacket 306. As such, the tip portion 304 can be more flexible than the body portion 302. In some examples, the tip portion 304 can be bounded to the distal end 302d of the body portion, e.g., via over-molding or the like.

As shown in FIG. 9, the body portion 302 of the dock sleeve 300 can also include an inner liner 310 to provide an inner layer against the docking device (e.g., 100) retained within the dock sleeve 300. The inner liner 310 can be made of various polymeric materials, such as PTFE. In some examples, the inner liner 310 can extend along and form an interior surface of the dock sleeve 300. In some examples, the inner liner 310 can extend into the middle section (e.g., 230) of the sleeve shaft. In some examples, as illustrated in FIG. 9, the inner liner 310 does not extend into the tip portion 304 of the dock sleeve 300.

In some examples, a hydrophilic coating 326 (also referred to as a “coated layer”), such as a hydrogel, can be applied on the outer surface of the dock sleeve 300. In some examples, the hydrophilic coating 326 can be configured to cover the outer surfaces of both the body portion 302 and the tip portion 304. The hydrophilic coating 326 can serve various purposes, such as allowing a sleeved docking device (e.g., 100) to navigate more easily around the native valve anatomy without significant friction. Additionally, the hydrophilic coating 326 can increase echogenicity, thus allowing visualization of the dock sleeve 300 using sonography.

In some examples, the dock sleeve 300 can include a radiopaque material to increase the ability to visualize the dock sleeve 300 during deployment of a docking device (e.g., 100). In some examples, the radiopaque material can be in the form of one or more marker bands 320 (similar to 231 as shown in FIGS. 8A-8B). In some examples, the radiopaque material can be embedded within the polymer jacket 306 and positioned proximal to the tip portion 304. In some examples, a metal braid or braided portion of the polymer jacket 306 can terminate a distance before a distal end of the marker band 320. In some examples, the radiopaque material of the marker band 320 can be a platinum-iridium marker. In other examples, the radiopaque marker can be formed on a section of flexible polymer loaded with Barium Sulphate (BaSO4), Bismuth Subcarbonate ((BiO)₂CO₃), Bismuth Oxychloride (BiOCl), or the like.

In some examples, the tip portion 304 of the dock sleeve 300 can be made from a polymeric material loaded with any one of the radiopaque material described above so as to enable the most distal edge (e.g., the distal end 304d) of the tip portion 304 to be visible under fluoroscopy.

While the polymer jacket, the support braid, the inner liner, the hydrophilic coating, and the radiopaque marker bands are described herein with reference to the dock sleeve 300, it is to be understood that the same or similar construction can be used for other dock sleeves, such as the dock sleeve 222 described above.

### Exemplary Tip Portion of Dock Sleeve

Referring to FIG. 9, the tip portion 304 can have a proximal end 304p connected to the distal end 302d of the body portion 302 and a distal end 304d located distal to the distal end 302d of the body portion 302 and defines the most distal edge of the dock sleeve 300. The tip portion 304 can, for example, help reduce the likelihood that the distal end of the delivery apparatus will snag or catch the native tissue (e.g., the chordae). The tip portion 304 can additionally or alternatively reduce the likelihood of blood flowing into the distal end of the delivery apparatus, which in turn can reduce or prevent thrombosis within the delivery apparatus.

In some examples, an axial length of the tip portion 304, measuring from the proximal end 304p to the distal end 304d, can range between about 1-4 mm. In one specific example, the axial length of the tip portion 304 is about 2 mm.

The tip portion 304 can taper radially inwardly from the proximal end 304p of the tip portion to the distal end 304d of the tip portion. The tapered tip portion 304 can facilitate atraumatic navigation around the native tissue at the implantation site for the docking device. As described above, a hydrophilic coating 326, such as hydrogel, can be applied on the outer surface of the dock sleeve 300. In some examples, the hydrophilic coating 326 can be configured to cover the outer surfaces of both the body portion 302 and the tip portion 304.

In some examples, as illustrated in FIG. 9, a cross-sectional profile of the tip portion 304 taken along a longitudinal axis 301 of the dock sleeve 300 can form a rounded shape between the proximal end 304p of the tip portion and the distal end 304d of the tip portion. In some examples, the rounded shape can be a semi-circle, or a partial circle with an arc angle less than 180 degrees. In some examples, the rounded shape can include two parallel lines at a proximal portion (e.g., defining a cylinder lumen) and a semi-circle at a distal portion (e.g., defining a half-sphere inner space). In some examples, the rounded shape can be a partial or half-ellipse, or the like.

The tip portion 304 can also have other shapes. In one example, as illustrated in FIG. 11A, the cross-sectional profile of the tip portion 304 taken along the longitudinal axis 301 of the dock sleeve 300 can have two edges 316 that linearly connect the proximal end 304p of the tip portion to the distal end 304d of the tip portion (i.e., each edge 316 can form a continuous, straight line connecting the proximal end 304p to the distal end 304d. The distal end 304d of the tip portion has a flat surface 318 that is perpendicular to the longitudinal axis 301 of the dock sleeve. In some examples, the flat surface 318 can be connected to the two edges 316 by rounded corners 317.

In another example, as illustrated in FIG. 11B, the cross-sectional profile of the tip portion 304 taken along the longitudinal axis 301 of the dock sleeve 300 can have a concave shape relative to a centroid 304c of the tip portion 304. As described herein, the centroid of the tip portion represents the arithmetic mean position of all the points in the tip portion. When the tip portion has a uniform density, the centroid of the tip portion is also the center of mass of the tip portion, which is typically located on the longitudinal axis 301.

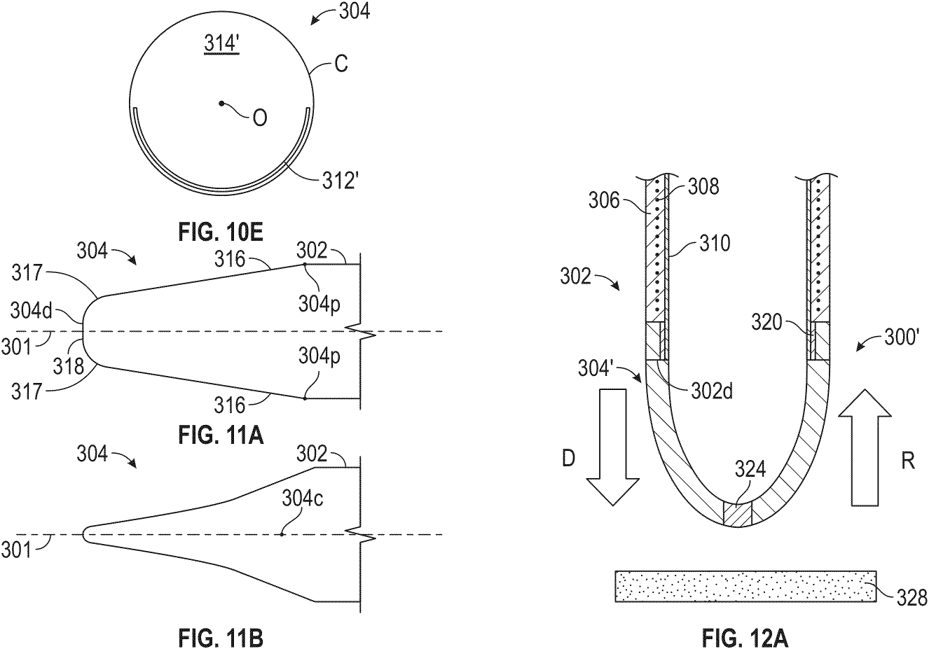

Referring to FIGS. 9 and 10A-10E, the tip portion of the dock sleeve can include one or more slits 312 defining one or more flaps 314. As illustrated in FIG. 9, the flaps 314 can move between a radially collapsed state (e.g., the tip portion in the radially collapsed state is shown in solid lines) and a radially expanded state (e.g., the tip portion in the radially expanded state is shown in dashed lines). In the radially collapsed state, the flaps 314 can collapse radially inwardly so as to cover a distal end (e.g., 102d) of the docking device (e.g., 100) retained within the dock sleeve 300 and occlude the lumen 305 of the body portion 302. In the radially expanded state, the flaps 314 can expand radially outwardly so as to allow the distal end (e.g., 102d) of the docking device (e.g., 100) to extend distally from the lumen 305 of the body portion 302 and beyond the tip portion 304 such that the distal end (e.g., 102d) of the docking device (e.g., 100) is uncovered by the dock sleeve 300.

As noted above, at least the main tube (e.g., 250) of a pusher shaft (e.g., 212) can extend through the sleeve shaft (e.g., 220) comprising the dock sleeve 300, and a distal end (e.g., 250d) of the main tube (e.g., 250) can press against the proximal end (e.g., 102p) of the docking device (e.g., 100) enclosed within the dock sleeve 300. Thus, after the docking device (e.g., 100) enclosed within the dock sleeve 300 is implanted at the target implantation site, the sleeve shaft (e.g., 220) can be retracted in the proximal direction relative to the docking device while holding the pusher shaft (e.g., 212) steady. As a result, the distal end (e.g., 102d) of the docking device (e.g., 100) can push the flaps 314 radially outwardly when it advances distally out of the dock sleeve 300 through the tip portion 304. In other words, when the distal end 302d of the body portion 302 is axially aligned with the distal end (e.g., 102d) of the docking device, the tip portion 304 is in the radially collapsed state (and the dock sleeve 300 is in the “covered state”), whereas when the distal end (e.g., 102d) of the docking device is disposed distal to the tip portion 304, the tip portion 304 is in the radially expanded state (and the dock sleeve 300 is in the “uncovered state”).

FIGS. 10A-10E show end views of the tip portion of a dock sleeve having various configurations of slits and flaps, according to certain examples. As shown, in the axially projected view, the proximal end (e.g., 304p) of the tip portion can define a circle C, and the distal-most point (e.g., 304d) of the tip portion can define a center O of the circle C.

In the examples shown in FIGS. 10B and 10D, the tip portion 304 has one slit 312 across the center O of the tip portion 304, thereby dividing the tip portion 304 into two equal sized flaps 314. In some examples, the single slit 312 does not need to cross the center O of the tip portion 304, thereby dividing the tip portion 304 into to unequal sized flaps 314. In the examples shown in FIGS. 10A and 10C, the tip portion 304 has two slits 312 dividing the tip portion 304 into four flaps 314. In the depicted examples, both slits 312 cross the center O of the tip portion 304 and are perpendicular to each other, thereby creating four equal sized flaps 314. In other examples, the two slits 312 may not be perpendicular to each other, and/or at least one of the slits 312 does not cross the center O of the tip portion 304, thereby creating four unequal sized flaps 314.

In the examples depicted in FIGS. 10A-10D, each slit 312 extends generally along a diameter of the circle C. For example, as shown in FIG. 10B, the slit 312 has two ends 312a, 312b which are located diametrically opposed to each other, and both ends 312a, 312b are adjacent to the edge of the circle C. In some examples, the length of the slit 312 (e.g., the distance between the ends 312a and 312b) can be about 50-100% of the diameter of the circle C. For example, the length of the slit 312 can be about 75%, 80%, 85%, 90%, 95, or 100% of the diameter of the circle C. When the length of the slit 312 equals to the diameter of the circle C, the ends (e.g., 312a, 312b) of the slit 312 are located on the edge of the circle C, i.e., the slit 312 extends to the proximal end (e.g., 304p) of the tip portion. In other examples, a slit may not extend edge-to-edge across the circle C. For example, a slit may extend along a radius of the circle C, i.e., the slit may extend from the center O of the tip portion to an edge (or to a point adjacent to the edge) of the circle C.

While the slits 312 shown in FIGS. 10A-10D are straight, the slit can also have a curved shape. For example, in the example shown in FIG. 10E, a C-shaped slit 312′ is located just inside and adjacent to the circle C, thereby defining only one flap 314′. In the depicted example, the C-shaped slit 312′ extends in a circumferential direction for about 180 degrees. In other examples, the curved slit 312′ can extend in the circumferential direction for more than or less than 180 degrees. In some examples, the curved slit 312′ may be spaced away from the circle C, e.g., adjacent to the center O, thus creating a pair of flaps located on both sides of the slit 312′. In some examples, more than one curved slit 312′, or a combination of curved slit(s) and straight slit(s) can be created on the tip portion.

In some examples, as shown in FIGS. 10C and 10D, the tip portion (e.g., 304) can have an aperture 322 around the center O, through which the longitudinal axis (e.g., 301) of the dock sleeve extends. In some examples, the diameter of the aperture 322 can be less than 0.4 mm. In some examples, the diameter of the aperture 322 can be less than 0.2 mm. In one particular example, the diameter of the aperture 322 can be about 0.1 mm.

As described herein, the width of the slit(s) (e.g., 312) and the size of the aperture (e.g., 322) (if the aperture is present) can be configured so that when the flaps (e.g., 314) are in the radially collapsed state, a flush fluid that flows through the dock sleeve and around the docking device (see, e.g., the flush fluid flow 264 in FIG. 7) can exit from the dock sleeve through such slit(s) and the aperture (if present) at a predetermined flow rate, while the flaps can still substantially occlude the lumen (e.g., 305) of the dock sleeve and cover the distal end (e.g., 102d) of the docking device (e.g., 100) retained within the lumen. The flush fluid can, for example, reduce thrombosis.

As described herein, the flaps can substantially occlude the lumen of the dock sleeve when the flaps cover at least 80%, or 85%, or 90%, or at least 95% of the area defined by the circle C (i.e., the cross-sectional area of the body portion of the dock sleeve taking in a direction that is perpendicular to the longitudinal axis of the dock sleeve).

In some examples, the tip portion (e.g., 304) has no aperture 322 and the flaps (e.g., 314) can cover 100% of the area defined by the circle C. As such, the flaps can completely occlude the lumen (e.g., 305) of the dock sleeve in the absence of flush fluid flowing through the dock sleeve. When there is a flush fluid flowing through the dock sleeve, the flush fluid can exert a pressure on the flaps (which are soft and flexible) in the distal direction, thus causing the flaps to radially expand slightly and open a small outlet (which can function like the aperture 322) for the flush fluid to drip out of the dock sleeve.

As noted above and shown in FIG. 9, the dock sleeve 300 can incorporate a radiopaque material in the form of one or more marker bands 320 embedded within the polymer jacket 306 and positioned proximal to the tip portion 304. In some examples, the dock sleeve 300 can include a radiopaque marker disposed on the tip portion 304. For example, a radiopaque marker 324 can be disposed at the distal end 304d (i.e., the distal-most area) of the tip portion 304. In some examples, the tip portion 304 can have a plurality of radiopaque markers, which may be uniformly or non-uniformly distributed on the plurality of flaps 314 of the tip portion 304. In some examples, a radiopaque marker can be configured to cover an entire area of the tip portion (e.g., the surface areas of all flaps).

### Exemplary Method of Creating Dock Sleeve

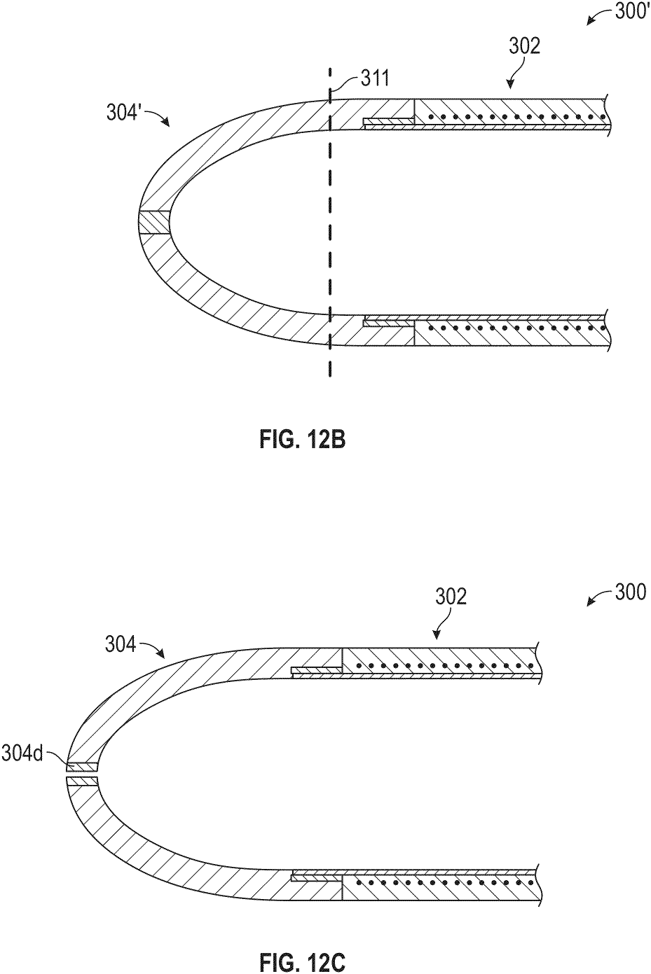

FIGS. 12A-12C show a method of creating the dock sleeve 300 described above, according to one example. In general, an intact dock sleeve 300′ can be initially created. The intact dock sleeve 300′ can have a body portion 302 and a tip portion 304′ similar to the dock sleeve 300 described above, except that the tip portion 304′ completely closes or seals a distal end of the body portion 302. Then a coating material can be added to the intact dock sleeve 300′ to create a coated layer 326 (see, e.g., FIG. 9). After the coating, one or more slits 312 and/or the aperture 322 (see, e.g., FIGS. 10A-10D) can be created on the tip portion, thereby creating the dock sleeve 300.

The intact dock sleeve 300′ can be created in a number of ways. For example, the body portion 302 and the tip portion 304′ can be created as a unitary piece if the tip portion 304′ is constructed of the same polymeric material as the polymer jacket 306 used to construct the body portion 302. In another example, the tip portion 304′, the material of which may be the same as or different from the body portion 302, may be attached to the distal end 302d of the body portion, e.g., by means of over-molding or similar techniques. As noted above, the polymer jacket 306 in the body portion 302 can be reinforced by a braided layer 308, and an inner liner 310 can be disposed on the interior surface of the body portion 302. The tip portion 304′ can be pre-shaped to a desired geometry (e.g., the rounded shape in FIG. 12A, or other shapes as shown in FIGS. 11A-11B). Alternatively, the tip portion 304′ can initially have a shape that is different from the desired geometry, and a tipping process (e.g., via thermoforming, etc.) can be applied to shape the tip portion 304′ to the desired geometry. In certain circumstances, before the tipping process, one or more radiopaque markers (e.g., 320, 324) can be disposed on the dock sleeve 300′, e.g., on the tip portion 304′ and/or the body portion 302, as described above. Incorporation of the radiopaque marker to the dock sleeve 300′ can be achieved by a variety of means, such as adhesion, embedding, molding, impregnation, or the like.

As illustrated in FIG. 12A, the intact dock sleeve 300′ (including both the tip portion 304′ and at least a portion of the body portion 302) can be dipped (as indicated by the arrow D) into a solution 328 comprising a liquified hydrophilic coating material, such as hydrogel. Then the intact dock sleeve 300′ can be removed (as indicated by the arrow R) from the solution 328 to allow the liquified coating material to cure. As such, the outer surface of the intact dock sleeve 300′ (including both the body portion 302 and the tip portion 304′) can be coated with the coating material to create a lubricious coated layer 326. Because the tip portion 304′ completely plugs or seals the distal end of the body portion 302, the hydrophilic coating material cannot enter the interior space of the dock sleeve 300′ through the tip portion 302′.

In some examples, in lieu of or in addition to the hydrophilic coating described above, a surface lubricant, e.g., silicone lubricant, can be applied to the outer surface of the intact dock sleeve 300′ (including both the body portion 302 and the tip portion 304′).

While dip coating has been shown in FIG. 12A as a means to create a hydrophilic coating for the intact dock sleeve 300′, alternative methods can be used to create the lubricious coated layer 326. For example, the coated layer 326 can be formed using an electrospinning technique, a centrifugal spinning technique, an atmospheric plasma spray technique, a spray coating technique, a melt-spinning technique, or the like.

After the outer surface of the intact dock sleeve 300′ has been coated with the hydrophilic coating material, one or more slits 312 can be created on the tip portion 304′, e.g., by using a sharp blade, or laser, or any other cutting means. For example, as described above with reference to FIGS. 10A-10D, at least one slit can cut open the tip portion along its radial diameter. In another example, two slits can cut across the tip portion and intersect with each other. As described above, the one or more slits 312 can create one or more flaps 314, which enables the tip portion 304 to stretch open when pressed upon by the distal end of the docking device. Each slit 312 can be cut to a predefined axial length from the distal tip, as noted above. For example, as shown in FIG. 12B, the dashed line 311 marks how far “into” (i.e., left to right in the depicted orientation) the tip portion 304 the slits 312 extend. As noted above, the ends (e.g., 312a, 312b) of slit 312 can extend to the proximal end 304p of the tip portion, or to positions adjacent to proximal end 304p of the tip portion.

Optionally, an aperture 322 can be created at the distal end 304d of the tip portion 304 along the longitudinal axis of the dock sleeve 300. The aperture 322 can be created by punching with a hole puncher, or cutting with a small cutting tool, or drilling with a drill bit, or by laser cutting, or the like. The slits 312 and the aperture 322 can be created simultaneously (e.g., as part of a single cutting process) or sequentially (e.g., in separate processes). After creating of the slits 312 (and optionally the aperture 322), the intact dock sleeve 300′ is converted to the dock sleeve 300, as shown in FIG. 12C.

### Exemplary Implantation Procedure

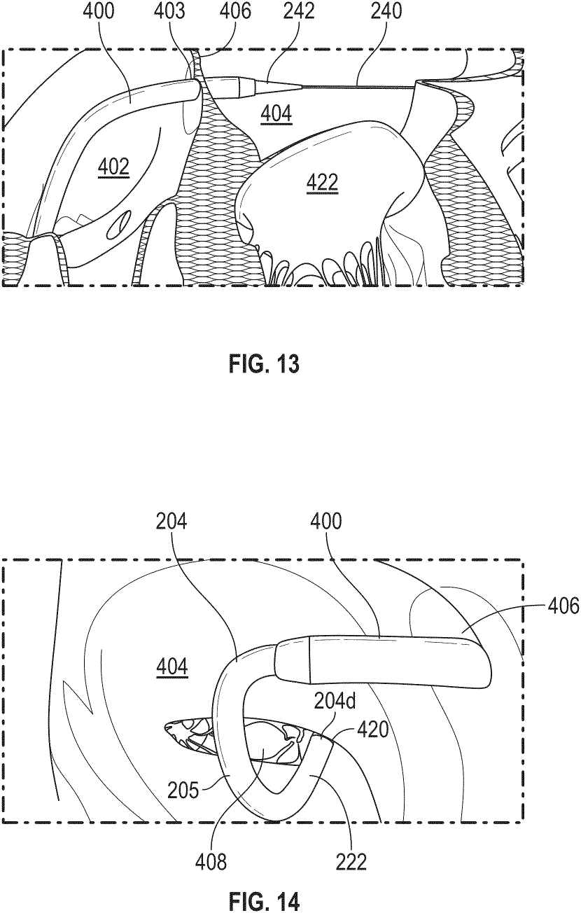

An example method of delivering a docking device (such as the docking device 100 described above) and implanting a prosthetic valve (such as the prosthetic valve 10 described above) within the docking device is illustrated in FIGS. 13-26. In this example, the target implantation site is at the native mitral valve 422. Following the same principle described herein, the same method or its variants can also be used for implantation of the docking device and the prosthetic valve at other target implantation sites.

FIG. 13 illustrates introducing a guiding catheter 400 into a patient’s heart over a previously inserted guidewire 240. Specifically, the guiding catheter 400 and the guidewire 240 are inserted from the right atrium 402 into the left atrium 404 through the interatrial septum 406. To facilitate navigation through the patient’s vasculature and transseptal insertion, a nosecone 242 having a tapered distal tip can be placed at a distal end of the guiding catheter 400. After the distal end of the guiding catheter 400 enters the left atrium 404, the nosecone 242 and the guidewire 240 can be retracted back into the guiding catheter 400, for example, by operating a handle connected to a proximal end of the guiding catheter 400. The guiding catheter 400 can remain in place (i.e., extending through the interatrial septum) so that the distal end of the guiding catheter 400 remains within the left atrium 404.

FIG. 14 illustrates introducing a delivery apparatus (such as the delivery apparatus 200 described above) through the guiding catheter 400. Specifically, the delivery sheath 204 can be inserted through a lumen of the guiding catheter 400 until the distal end portion 205 of the delivery sheath 204 extends distally out of the distal end of the guiding catheter 400 and into the left atrium 404.

As described above, the delivery apparatus 200 can have a sleeve shaft 220 and a pusher shaft 212, both of which can extend through a lumen of the delivery sheath 204. In FIGS. 15-16, the distal end portion of the sleeve shaft 220 is shown to have a dock sleeve 222 which retains the docking device 100, although it is to be understood that other examples of dock sleeve (e.g., 300) can be similarly used. As described herein, the dock sleeve 222 can be retained within the distal end portion 205 of the delivery sheath 204.

As described above, the distal end portion 205 of the delivery sheath 204 can be steerable, for example, by operating a knob located on the handle assembly 202. Because the dock sleeve 222 and the docking device 100 are also flexible, flexing of the distal end portion 205 of the delivery sheath 204 can also cause flexing of the dock sleeve 222 and the docking device 100 retained therein. As shown in FIG. 14, the distal end portion 205 of the delivery sheath 204 (along with the dock sleeve 222 retaining the docking device 100) can be flexed in desired angular directions so that the distal end 204d of the delivery sheath 204 can extend through the native mitral valve annulus 408 at a location adjacent the posteromedial commissure 420 and into the left ventricle 414.

FIG. 15 illustrates deployment of the docking device 100 at the mitral valve location. As shown, a distal portion of the docking device 100, which includes the leading turn 106 and the central region 108 of the coil, can be deployed out of the distal end 204d of the delivery sheath 204 and extend into the left ventricle 414. Note that the deployed distal portion of the docking device 100 is still covered by the dock sleeve 222. This can be achieved, for example, by pushing both the pusher shaft 212 and the sleeve shaft 220 in the distal direction while holding the delivery sheath 204 steady, thus causing the distal portion of the docking device 100 to extend distally out of the delivery sheath 204 while it remains to be covered by the dock sleeve 222. As noted above, the distal section 258 of the pusher shaft 212 can also be flexible. Thus, the distal section 258 of the pusher shaft 212 can also flex and/or bend along the flexed or curved distal end portion 205 of the delivery sheath 204 when pushing the pusher shaft 212 in the distal direction to deploy the docking device 100.

Not being restrained by the distal end portion 205 of the delivery sheath 204, the distal portion of the docking device 100 can move from the delivery configuration to the deployed (helical) configuration. Specifically, as shown in FIG. 15, the coil of the docking device 100 (covered by the dock sleeve 222) can form the leading turn 106 extending into the left ventricle 414, as well as a plurality of functional turns in the central region 108 that wrap around the native leaflets 410 of the native valve and the chordae tendineae 412 connected thereto.

Because the dock sleeve 222 has a lubricious surface, it can prevent the first cover 112 (which surrounds the coil 102 of the docking device) from directly contacting and catching (or getting stuck with) the native tissue, and facilitate the covered docking device 100 to encircle the native anatomy. In addition, the soft tip portion 223 (which can have a taper shape) of the dock sleeve 222 can also facilitate atraumatic encircling around the native tissue. As noted above, a flush fluid (see, e.g., 264 in FIG. 7) can flow through the dock sleeve 222 and around the docking device 100 to prevent thrombosis on and around the docking device 100 and other concentric parts of the delivery apparatus 200 during deployment of the docking device 100.

Further, when the distal end portion of the sleeve shaft 220 comprises the dock sleeve 300, the tip portion 304 can remain in the radially collapsed state (i.e., the flaps 314 can occlude the lumen of the body portion 302 and cover the distal end of the docking device 100) during the procedure when the distal portion of the docking device 100 is pushed out of the delivery sheath 204 and encircles the native tissue, thereby further preventing bodily fluid (e.g., blood) from entering the lumen of the dock sleeve 300 and coagulate around the docking device 100 or other parts of the delivery apparatus 200.

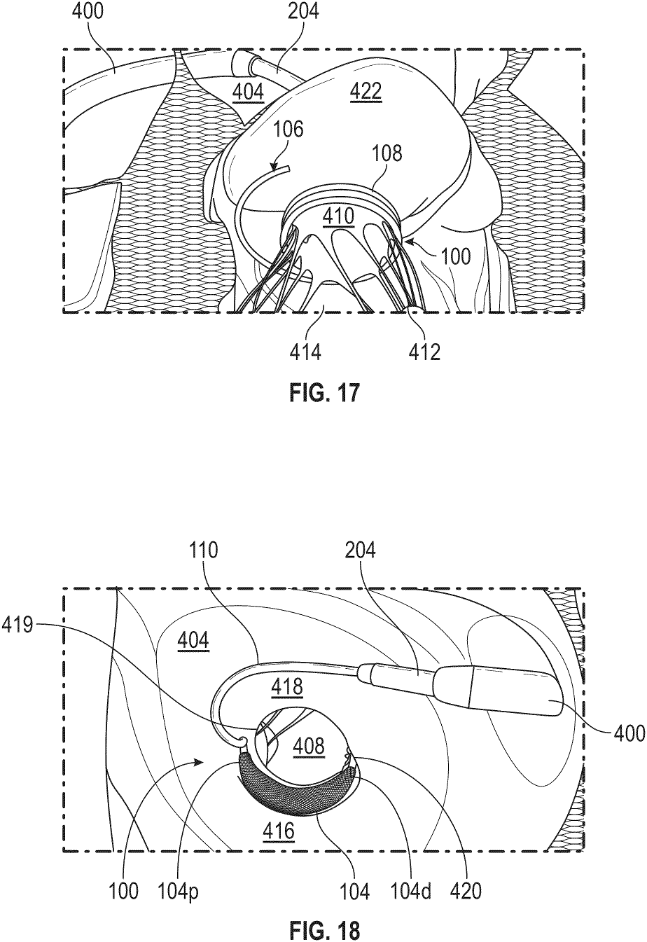

As shown in FIG. 16, after the functional turns of the docking device 100 successfully wraps round the native leaflets 410 and the chordae tendineae 412, the dock sleeve 222 can be retracted in a proximal direction relative to the docking device 100. This can be achieved, for example, by pulling the sleeve shaft 220 in the proximal direction while holding the pusher shaft 212 steady so that its distal end can press against the proximal end of the docking device 100, as described above with reference to FIG. 6B. As noted above, the dock sleeve 222 can be retracted back into the delivery sheath 204. FIG. 17 shows the docking device 100, which is uncovered by the dock sleeve 222, encircles the native tissue.

FIG. 18 illustrates stabilizing the docking device 100 at the atrial side. As shown, the delivery sheath 204 can be retracted into the guiding catheter 400 so that the atrial side (i.e., the proximal portion) of the docking device 100, including the stabilization turn 110 of the coil can be exposed. The stabilization turn 110 can be configured to provide one or more points or regions of contact between the docking device 100 and the left atrial wall, such as at least three points of contact in the left atrium or complete contact on the left atrial wall. The stabilization turn 110 can be flared out or biased toward both the posterior wall 416 and anterior wall 418 of the left atrium so as to prevent the docking device 100 from falling into the left ventricle prior to deployment of a prosthetic valve therein. As shown, the guard member 104 of the docking device 100 can be configured to contact the native annulus in the left atrium to create a sealed and atraumatic interface between the docking device 100 and the native tissue. The proximal end portion 104p of the guard member can be configured to positioned adjacent the anterolateral commissure 419 of the native valve. The distal end portion 104d of the guard member can be disposed adjacent a posteromedial commissure 420 of the native valve so that leakage at that location can be prevented or reduced.

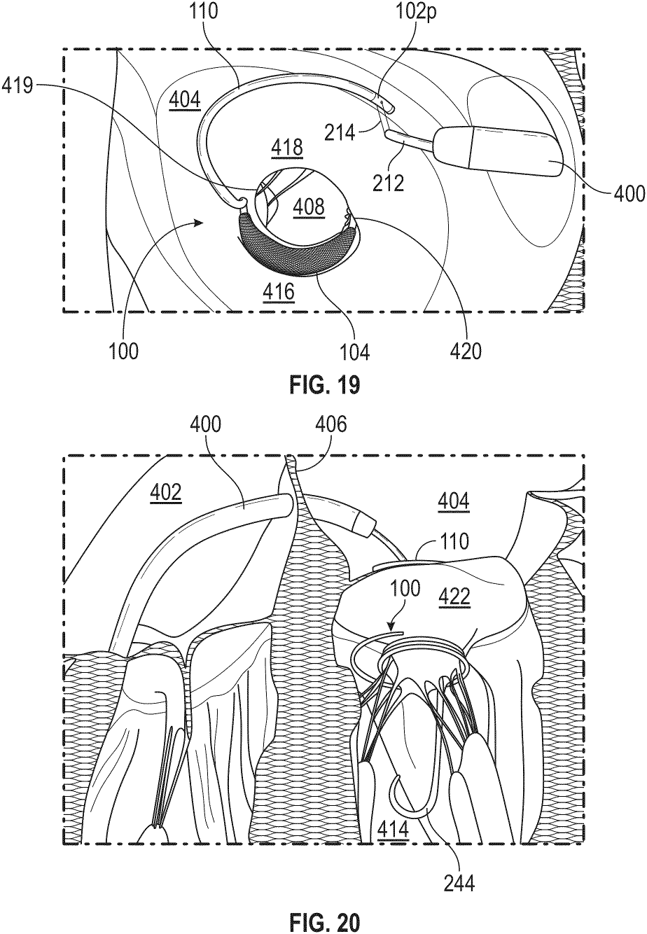

FIG. 19 illustrates the fully deployed docking device 100. The release suture 214, which extends through the pusher shaft 212 and connects the proximal end 102p of the coil to the suture lock assembly 216, can then be cut so that the docking device 100 can be released from the delivery apparatus 200. The delivery apparatus 200 can then be removed from the guiding catheter 400 to prepare for implantation of a prosthetic valve.

FIG. 20 illustrates inserting a guide wire catheter 244 through the guiding catheter 400, across the native mitral valve annulus through the docking device 100, and into the left ventricle 414.

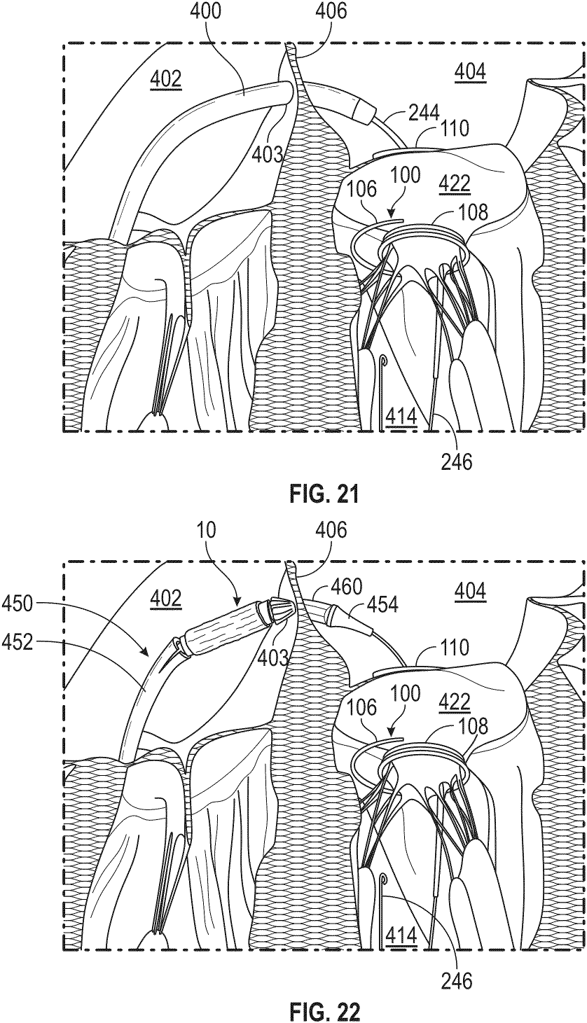

FIG. 21 illustrates inserting a valve guidewire 246 into the left ventricle 414 through an inner lumen of the guide wire catheter 244. The guidewire catheter 244 can then be retracted back into the guiding catheter 400, and the guiding catheter 400 and the guidewire catheter 244 can be removed, leaving the valve guidewire 246 in place.

FIG. 22 illustrates transseptal delivery of a prosthetic valve (such as the prosthetic valve 10) into the left atrium 404. A prosthetic valve delivery apparatus 450 can be introduced over the guidewire 246. During delivery, the prosthetic valve 10 can be crimped over a deflated balloon 460 located between a distal end of an outer shaft 452 and a nosecone 454 of the delivery apparatus 450. In some examples, before transseptal delivery of the prosthetic valve 10, the hole 403 on the interatrial septum 406 can be further dilated by inserting a balloon catheter through the hole 403 and radially expanding a balloon mounted on the balloon shaft.

FIG. 23 illustrates placing the prosthetic valve 10 within the docking device 100. Specifically, the prosthetic valve 10 can be positioned within and substantially coaxial with the functional turns in the central region 108 of the docking device 100. In some examples, the outer shaft 452 can be slightly retracted so that the balloon 460 is located outside the outer shaft 452.

FIG. 24 illustrates radial expansion of the prosthetic valve 10 within the docking device 100. Specifically, the balloon 460 can be radially inflated by injecting an inflation fluid into the balloon through the delivery apparatus 450, thereby causing radial expansion of the prosthetic valve 10. As the prosthetic valve 10 is radially expanded within the central region 108 of the coil, the functional turns in the central region 108 can be further radially expanded (i.e., the coil 102 of the docking device can move from the first radially expanded configuration to the second radially expanded configuration, as described above). To compensate for the increased diameter of the function turns, the leading turn 106 can be retracted in the proximal direction and become a part of the functional turn in the central region 108. In other words, the diameter of the leading turn 106 is reduced when the prosthetic valve 10 is expanded.

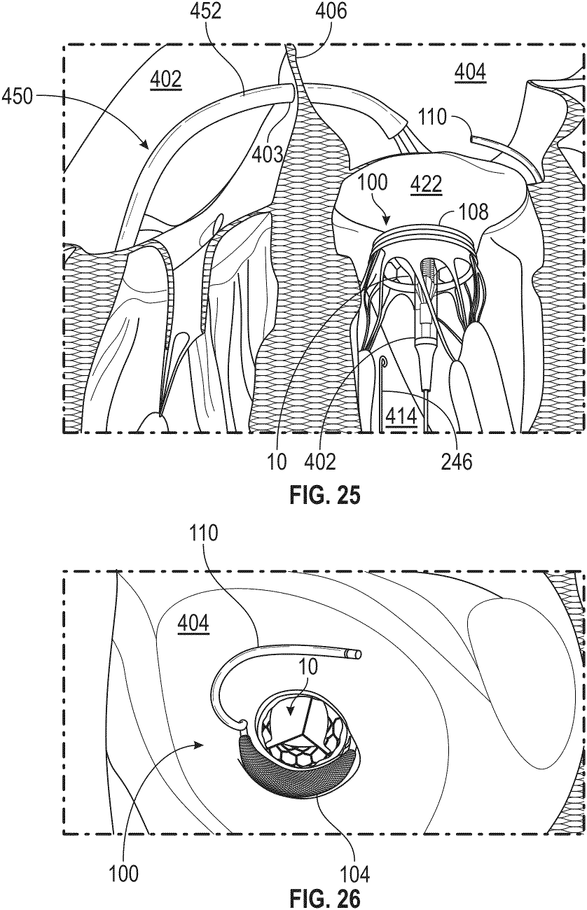

FIG. 25 illustrates deflating the balloon 460 after radial expansion of the prosthetic valve 10 within the docking device 100. The balloon 460 can be deflated by withdrawing the inflation fluid out of the balloon through the delivery apparatus 450. The delivery apparatus 450 can then be retracted out of the patient’s vasculature, and the guidewire 246 can also be removed.

FIG. 26 illustrates the final disposition of the docking device 100 at the mitral valve and the prosthetic valve 10 received within the docking device 100. As described above, the radial tension between the prosthetic valve 10 and the central region 108 of the docking device can securely hold the prosthetic valve 10 in place. In addition, the guard member 104 can act as an improved seal between the docking device 100 and the prosthetic valve 10 disposed therein to prevent paravalvular leakage around the prosthetic valve 10.

Although in the method described above, the prosthetic valve 10 is radially expanded using the inflatable balloon 460, it is to be understood that alternative methods can be used to radially expand the prosthetic valve 10.

For example, in some examples, the prosthetic valve can be configured to be self-expandable. During delivery, the prosthetic valve can be radially compressed and retained within a valve sheath located at a distal end portion of a delivery apparatus. When the valve sheath is disposed within the central region 108 of the docking device, the valve sheath can be retracted to expose the prosthetic valve, which can then self-expand and securely engage with the central region 108 of the docking device. Additional details regarding exemplary self-expandable prosthetic valves and the related delivery apparatus/catheters/systems are described in U.S. Pat. Nos. 8,652,202 and 9155,619, the entirety of which is incorporated by reference herein.

In another example, in certain examples, the prosthetic valve can be mechanically expanded. Specifically, the prosthetic valve can have a frame comprising a plurality of struts that are connected to each other such that an axial force applied to the frame (e.g., pressing an inflow and an outflow end of the frame in toward each other or pulling the inflow end and the outflow end of the frame away from each other) can cause the prosthetic valve to radially expand or compress. Additional details regarding exemplary mechanically-expandable prosthetic valves and the related delivery apparatus/catheters/systems are described in U.S. Pat. Application Publication No. 2018/0153689 and PCT Patent Application Publication No. WO/2021/188476, the entirety of which are incorporated by reference herein.

### Exemplary Embodiments

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus comprising: a dock sleeve comprising a body portion and a tip portion located at a distal end of the body portion and configured to be axially movable relative to a docking device for a prosthetic implant, wherein the body portion comprises a lumen configured to receive the docking device therein, wherein the tip portion comprises one or more slits defining one or more flaps, wherein the one or more flaps are movable between a radially collapsed state and a radially expanded state, wherein in the radially collapsed state, the one or more flaps cover a distal end of the docking device and occlude the lumen of the body portion, and wherein in the radially expanded state, the one or more flaps allow the distal end of the docking device to extend distally from the lumen of the body portion and beyond the tip portion such that the distal end of the docking device is uncovered by the dock sleeve.

Example 2. The delivery apparatus of any example herein, particularly example 1, wherein the tip portion comprises one C-shaped slit defining one flap.

Example 3. The delivery apparatus of any example herein, particularly example 1, wherein the tip portion comprises one slit dividing the tip portion into two flaps.

Example 4. The delivery apparatus of any example herein, particularly example 1, wherein the tip portion comprises two crossing slits dividing the tip portion into four flaps.

Example 5. The delivery apparatus of any example herein, particularly any one of examples 1-4, wherein the tip portion comprises an aperture located along a longitudinal axis of the dock sleeve.

Example 6. The delivery apparatus of any example herein, particularly any one of examples 1-5, wherein the tip portion comprises a proximal end connected to the distal end of the body portion and a distal end located distal to the distal end of the body portion.

Example 7. The delivery apparatus of any example herein, particularly example 6, wherein an axial distance from the proximal end of the tip portion to the distal end of the tip portion is about 2 mm.

Example 8. The delivery apparatus of any example herein, particularly any one of examples 6-7, wherein the tip portion tapers radially inwardly from the proximal end of the tip portion to the distal end of the tip portion.

Example 9. The delivery apparatus any example herein, particularly of example 8, wherein a cross-sectional profile of the tip portion taken along a longitudinal axis of the dock sleeve forms a rounded shape between the proximal end of the tip portion and the distal end of the tip portion.

Example 10. The delivery apparatus of any example herein, particularly example 9, wherein the rounded shape is a semi-circle.

Example 11. The delivery apparatus of any example herein, particularly example 8, wherein a cross-sectional profile of the tip portion taken along a longitudinal axis of the dock sleeve comprises two edges that linearly connect the proximal end of the tip portion to the distal end of the tip portion.

Example 12. The delivery apparatus of any example herein, particularly example 11, wherein the distal end of the tip portion has a flat surface that is perpendicular to the longitudinal axis of the dock sleeve.

Example 13. The delivery apparatus of any example herein, particularly example 12, wherein the flat surface is connected to the two edges by rounded corners.

Example 14. The delivery apparatus of any example herein, particularly example 8, wherein a cross-sectional profile of the tip portion taken along a longitudinal axis of the dock sleeve has a concave shape relative to a centroid of the tip portion.

Example 15. The delivery apparatus of any example herein, particularly any one of examples 1-14, wherein the dock sleeve comprises one or more radiopaque markers.

Example 16. The delivery apparatus of any example herein, particularly example 15, wherein at least one radiopaque marker is disposed on the tip portion.

Example 17. The delivery apparatus of any example herein, particularly example 16, wherein the at least one radiopaque marker is disposed at a distal-most area of the tip portion.

Example 18. The delivery apparatus of any example herein, particularly any one of examples 16-17, wherein the at least one radiopaque marker is one of a plurality of radiopaque markers that are uniformly distributed on the tip portion.

Example 19. The delivery apparatus of any example herein, particularly any one of examples 16-17, wherein the at least one radiopaque marker covers an entire area of the tip portion.

Example 20. The delivery apparatus of any example herein, particularly example 15, wherein at least one radiopaque marker is disposed at the distal end of the body portion.

Example 21. The delivery apparatus of any example herein, particularly any one of examples 1-20, wherein the tip portion comprises a polymeric material.

Example 22. The delivery apparatus of any example herein, particularly example 21, wherein the polymeric material comprises thermoplastic elastomers.

Example 23. The delivery apparatus of any example herein, particularly any one of examples 1-22, wherein the body portion of the dock sleeve comprises a polymer jacket and an inner liner disposed over an inner surface of the polymer jacket, wherein the inner liner defines an interior surface of at least a section of the body portion.

Example 24. The delivery apparatus of any example herein, particularly example 23, wherein the inner liner comprises a polymeric material.

Example 25. The delivery apparatus of any example herein, particularly example 24, wherein the polymeric material comprises PTFE.

Example 26. The delivery apparatus of any example herein, particularly any one of examples 23-25, wherein the polymer jacket comprises an elastomeric material and a support layer.

Example 27. The delivery apparatus of any example herein, particularly example 26, wherein the support layer comprises a metal braid.

Example 28. The delivery apparatus of any example herein, particularly any one of examples 1-27, wherein an outer surface of the body portion comprises a hydrophilic coating.

Example 29. The delivery apparatus of any example herein, particularly example 28, wherein the hydrophilic coating comprises hydrogel.

Example 30. The delivery apparatus of any example herein, particularly any one of examples 1-27, wherein an outer surface of the tip portion comprises a hydrophilic coating.

Example 31. The delivery apparatus of any example herein, particularly any one of examples 1-29, further comprising a pusher shaft configured to push the docking device in a distal direction so that the tip portion can move from the radially collapsed state to the radially expanded state when retracting the dock sleeve in a proximal direction while holding the pusher shaft steady, thereby pushing the docking device out of the dock sleeve through the tip portion.

Example 32. The delivery apparatus of any example herein, particularly example 31, wherein a distal end of the pusher shaft is configured to be inserted into a lumen of the dock sleeve and press against a proximal end of the docking device.

Example 33. The delivery apparatus of any example herein, particularly any one of examples 31-32, further comprising a delivery sheath, wherein the dock sleeve is a distal end portion of a sleeve shaft, wherein the sleeve shaft and the pusher shaft are coaxial with each other and extend through a lumen of the delivery sheath.

Example 34. The delivery apparatus of any example herein, particularly example 33, wherein a distal end portion of the delivery sheath is configured to surround the dock sleeve and retain the docking device in a substantially straight configuration.

Example 35. The delivery apparatus of any example herein, particularly example 34, wherein the delivery sheath is configured to be axially movable relative to the sleeve shaft and the pusher shaft such that when the dock sleeve and the docking device are removed from the distal end portion of the delivery sheath, the docking device can change from the substantially straight configuration to a helical configuration while the body portion of the dock sleeve remains an outer surface of the docking device.

Example 36. The delivery apparatus of any example herein, particularly any one of examples 33-35, wherein the pusher shaft and the sleeve shaft are configured to move together in an axial direction with the docking device when deploying the docking device from the delivery sheath.

Example 37. The delivery apparatus of any example herein, particularly any one of examples 33-36, further comprising a handle connected to a proximal end portion of the delivery sheath, a proximal end portion of the sleeve shaft, and a proximal end portion of the pusher shaft.

Example 38. The delivery apparatus of any example herein, particularly example 37, wherein the handle comprises a steering member configured to adjust a curvature of the distal end portion of the delivery sheath.

Example 39. The delivery apparatus of any example herein, particularly any one of examples 37-38, wherein the handle comprises one or more flushing ports configured to supply flush fluid to one or more lumens formed between the docking device, the sleeve shaft, the pusher shaft, and the delivery sheath.

Example 40. The delivery apparatus of any example herein, particularly any one of examples 31-39, wherein the pusher shaft comprises a stop element configured to limit proximal movement of the sleeve shaft relative to the pusher shaft.

Example 41. A dock sleeve for a delivery apparatus configured to implant a docking device, the dock sleeve comprising: a body portion and a tip portion located at a distal end of the body portion, wherein the dock sleeve is configured to be axially movable relative to the docking device, wherein the body portion is configured to cover at least a distal portion of the docking device when the distal end of the body portion axially aligns with a distal end of the docking device, wherein the tip portion is movable between a radially collapsed state and a radially expanded state, wherein when the body portion covers the distal portion of the docking device, the tip portion in the radially collapsed state covers the distal end of the docking device, and the tip portion in the radially expanded state allows the distal end of the docking device to move distally relative to the distal end of the body portion.

Example 42. A dock sleeve for implanting a docking device at a native valve, the dock sleeve comprising: a body portion and a tip portion located at a distal end of the body portion, wherein the dock sleeve is configured to be axially movable relative to the docking device, wherein the body portion is configured to cover at least a distal portion of the docking device when the distal end of the body portion axially aligns with a distal end of the docking device, wherein the tip portion comprises one or more slits dividing the tip portion into one or more flaps, wherein when the body portion covers the distal portion of the docking device, the one or more flaps can collapse radially inwardly so as to cover the distal end of the docking device and can expand radially outwardly when the distal end of the docking device is advanced distally through the tip portion.

Example 43. An implant assembly comprising: a docking device configured to be implanted at a native annulus of a patient, and a dock sleeve comprising a body portion and a tip portion located at a distal end of the body portion, wherein the dock sleeve is configured to cover the docking device during one or more portions of a delivery procedure and to be axially movable relative to the docking device such that the docking device can be exposed from the dock sleeve, wherein the body portion is configured to cover at least a distal portion of the docking device when the distal end of the body portion axially aligns with a distal end of the docking device, wherein the tip portion is movable between a radially collapsed state and a radially expanded state, wherein when the distal end of the body portion is axially aligned with the distal end of the docking device, the tip portion is in the radially collapsed state, and wherein when the distal end of the docking device is disposed distal to the tip portion, the tip portion is in the radially expanded state.

Example 44. The implant assembly of any example herein, particularly example 43, wherein the docking device comprises a coil configured to surround native tissue when deployed at the native annulus.

Example 45. An implant assembly comprising: a radially expandable and compressible prosthetic valve; a docking device configured to receive the prosthetic valve, wherein the prosthetic valve is configured to be radially expandable within the docking device; and a dock sleeve configured to be axially movable relative to the docking device, wherein the dock sleeve comprises a body portion and a tip portion located at a distal end of the body portion, wherein the body portion is configured to cover at least a distal portion of the docking device when the distal end of the body portion aligns with a distal end of the docking device, wherein the tip portion is movable between a radially collapsed state and a radially expanded state, wherein when the body portion covers the distal portion of the docking device, the tip portion in the radially collapsed state covers the distal end of the docking device, and the tip portion in the radially expanded state allows the distal end of the docking device to move distally relative to the distal end of the body portion so as to be uncovered by the dock sleeve.

Example 46. An implant assembly comprising: a docking device configured to surround native tissue at an implantation site of a patient; a dock sleeve configured to cover at least a distal portion and a distal end of the docking device when the docking device is delivered to the implantation site and surrounds the native tissue; and a pusher shaft configured to push the docking device in a distal direction relative to the dock sleeve so that a distal end of the dock sleeve is pressed open to allow the distal portion of the docking device to move out of the dock sleeve when retracting the dock sleeve in a proximal direction while holding the pusher shaft steady or pushing the pusher shaft in a distal direction while holding the dock sleeve steady.

Example 47. A delivery apparatus for implanting a docking device at a native valve, the delivery apparatus comprising: a dock sleeve configured to cover at least a distal portion and a distal end of the docking device when the docking device is delivered to the native valve; and

a pusher shaft configured to push the docking device in a distal direction relative to the dock sleeve so that a distal end of the dock sleeve is pressed open to allow the distal end of the docking device to move out of the dock sleeve when retracting the dock sleeve in a proximal direction while holding the pusher shaft steady or pushing the pusher shaft in a distal direction while holding the dock sleeve steady.

Example 48. A dock sleeve for implanting a docking device at a native valve, the dock sleeve comprising: a body portion and a tip portion located at a distal end of the body portion, wherein the dock sleeve is configured to be movable between a covered state and an uncovered state, wherein when the dock sleeve is in the covered state, the body portion covers at least a distal portion of the docking device and the tip portion covers a distal end of the docking device, wherein when the dock sleeve is in the uncovered state, the distal end of the docking device extends out of the dock sleeve through the tip portion of the dock sleeve.

Example 49. An implant assembly comprising: a docking device configured to be implanted at an implantation site of a patient; and a dock sleeve configured to be movable between a covered state and an uncovered state, wherein when the dock sleeve is in the covered state, the dock sleeve covers at least a distal portion and a distal end of the docking device, wherein when the dock sleeve is in the uncovered state, at least a distal end of the docking device extends out of the dock sleeve through the distal end of the dock sleeve.

Example 50. A delivery apparatus for implanting a docking device at a native valve, the delivery apparatus comprising: a dock sleeve configured to be movable between a covered state and an uncovered state, wherein when the dock sleeve is in the covered state, the dock sleeve covers at least a distal portion and a distal end of the docking device, wherein when the dock sleeve is in the uncovered state, at least a distal end of the docking device extends out of the dock sleeve through the distal end of the dock sleeve.

Example 51. A method of creating a dock sleeve configured to hold a docking device, the method comprising: creating a dock sleeve comprising a body portion and a tip portion, wherein the tip portion completely closes a distal end of the body portion; adding a coating material to the dock sleeve; and creating at least one slit on the tip portion.

Example 52. The method of any example herein, particularly example 51, wherein creating the dock sleeve comprises attaching the tip portion to the distal end of the body portion.

Example 53. The method of any example herein, particularly example 52, wherein attaching the tip portion to the distal end of the body portion comprises over-molding the tip portion to the distal end of the body portion.

Example 54. The method of any example herein, particularly any one of examples 51-53, wherein adding the coating material to the dock sleeve comprises coating at least a portion of an outer surface of the body portion and an outer surface of the tip portion with the coating material.

Example 55. The method of any example herein, particularly any one of examples 51-54, wherein the coating material is hydrophilic.

Example 56. The method of any example herein, particularly example 55, wherein the coating material comprises hydrogel.

Example 57. The method of any example herein, particularly any one of examples 51-56, wherein adding the coating material to the dock sleeve comprises dipping the dock sleeve to a solution of the coating material.

Example 58. The method of any example herein, particularly any one of example 51-56, wherein adding the coating material to the dock sleeve comprises depositing the coating material to an outer surface of the dock sleeve through electrospinning.

Example 59. The method of any example herein, particularly any one of examples 51-58, wherein creating the at least one slit on the tip portion comprises cutting open along a diameter of the tip portion.

Example 60. The method of any example herein, particularly any one of examples 51-59, wherein the at least one slit is a first slit, wherein the method further comprises cutting a second slit intersecting the first slit.

Example 61. The method of any example herein, particularly example 60, wherein the second slit is perpendicular to the first slit.

Example 62. The method of any example herein, particularly any one of examples 51-61, wherein creating the at least one slit comprises laser cutting the tip portion.

Example 63. The method of any example herein, particularly any one of examples 51-61, wherein creating the at least one slit comprises cutting the tip portion with a blade.

Example 64. The method of any example herein, particularly any one of examples 51-63, further comprising creating an aperture at a center of the tip portion.

Example 65. The method of any example herein, particularly example 64, wherein creating the aperture comprises punching the tip portion with a hole puncher.

Example 66. The method of any example herein, particularly example 64, wherein creating the aperture comprises laser cutting the tip portion.

Example 67. The method of any example herein, particularly any one of examples 51-66, wherein creating the dock sleeve comprises disposing one or more radiopaque markers on the dock sleeve.

Example 68. The method of any example herein, particularly example 67, wherein disposing the one or more radiopaque markers comprises disposing at least one radiopaque marker on the tip portion.

Example 69. The method of any example herein, particularly example 67, wherein disposing the one or more radiopaque markers comprises disposing at least one radiopaque marker at the distal end of the body portion.

Example 70. The method of any example herein, particularly any one of examples 51-66, wherein creating the dock sleeve comprises disposing an inner liner over an interior surface of at least a section of the body portion, wherein the inner liner comprises a polymeric material.

Example 71. A method for implanting a docking device at a target implantation site, the method comprising: deploying the docking device retained within a dock sleeve at the target implantation site, wherein at least a distal portion of the docking device is covered by a body portion of the dock sleeve and a distal end of the docking device is covered by a tip portion of the dock sleeve, wherein the tip portion is located at a distal end of the body portion; and removing the dock sleeve from the docking device so that the distal portion and the distal end of the docking device are exposed.

Example 72. The method of any example herein, particularly example 71, wherein the target implantation site is a native mitral valve, wherein deploying the docking device comprises creating a hole on a septum between a left atrium and right atrium, and navigating the docking device from the right atrium, through the hole on the septum, into the left atrium, and into a left ventricle through the native mitral valve.

Example 73. The method of any example herein, particularly any one of examples 71-72, wherein deploying the docking device comprises navigating a delivery sheath to a location adjacent the target implantation site, wherein a distal end portion of the delivery sheath surrounds the dock sleeve and retain the docking device in a substantially straight configuration.

Example 74. The method of any example herein, particularly example 73, where deploying the docking device further comprises pushing a distal portion of the docking device and the dock sleeve out of a distal end of the delivery sheath to allow the distal portion of the docking device to move from the substantially straight configuration to a helical configuration comprising one or more turns configured to wrap around native tissues at the target implantation site.

Example 75. The method of any example herein, particularly example 74, wherein deploying the docking device further comprises retracting the delivery sheath in a proximal direction relative to the docking device so as to expose a proximal portion of the docking device and allow the proximal portion of the docking device to move from the substantially straight configuration to a helical configuration.

Example 76. The method of any example herein, particularly example 75, wherein deploying the docking device further comprises anchoring the proximal portion of the docking device at a surrounding native wall adjacent the target implantation site.

Example 77. The method of any example herein, particularly any one of examples 73-76, wherein deploying the docking device further comprises monitoring a position of a radiopaque marker on the dock sleeve under fluoroscopy.

Example 78. The method of any example herein, particularly any one of examples 73-77, wherein deploying the docking device further comprises monitoring a position of a radiopaque marker on the docking device under fluoroscopy.

Example 79. The method of any example herein, particularly any one of examples 73-78, wherein navigating the delivery sheath comprises actuating a steering mechanism to adjust a curvature of the distal end portion of the delivery sheath.

Example 80. The method of any example herein, particularly any one of examples 73-79, further comprising releasing the docking device from the delivery sheath.

Example 81. The method of any example herein, particularly any one of examples 73-80, wherein deploying the docking device comprises pushing the delivery sheath out of a distal end of a delivery sheath, wherein the delivery sheath is steerable.

Example 82. The method of any example herein, particularly any one of examples 71-81, wherein removing the dock sleeve from the docking device comprises retracting the dock sleeve in a proximal direction relative to the docking device so that the distal end of the docking device extends out of the dock sleeve through tip portion of the dock sleeve.

Example 83. The method of any example herein, particularly any one of examples 71-82, wherein the tip portion comprises one or more flaps that are movable between a radially collapsed state and a radially expanded state, wherein in the radially collapsed state, the one or more flaps cover the distal end of the docking device when the distal portion of the docking device is covered by the body portion, and wherein in the radially expanded state, the one or more flaps allow the distal end of the docking device to extend distally beyond the tip portion such that the distal end of the docking device is uncovered by the dock sleeve.

Example 84. A method for implanting a prosthetic valve, the method comprising: deploying a docking device retained within a dock sleeve at a native valve, wherein at least a distal portion and a distal end of the docking device are covered by the dock sleeve; removing the dock sleeve from the docking device so that the distal portion and the distal end of the docking device are exposed; and deploying the prosthetic valve within the docking device.

Example 85. The method of any example herein, particularly example 84, wherein the docking device comprises a coil having a stabilization turn and one or more functional turns distal to the stabilization turn, wherein deploying the docking device at the native valve comprises wrapping around leaflets of the native valve with the one or more functional turns of the coil and resting the stabilization turn of the coil against a native wall around the native valve.

Example 86. The method of any example herein, particularly example 85, wherein the docking device comprises a guard member covering at least a portion of the stabilization turn.

Example 87. The method of any example herein, particularly any one of examples 85-86, wherein deploying the prosthetic valve comprises placing the prosthetic valve in a radially compressed state within the one or more functional turns of the coil and radially expanding the prosthetic valve to a radially expanded state, wherein radially expanding the prosthetic valve causes radial expansion of the one or more functional turns of the coil.

Example 88. The method of any example herein, particularly any one of examples 84-87, wherein the docking device is movable between a substantially straight configuration and a helical configuration, wherein the dock sleeve is configured to retain the docking device when the docking device moves from the substantially straight configuration to the helical configuration.

### Exemplary Alternatives

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

1. A delivery apparatus comprising:
a dock sleeve comprising a body portion and a tip portion located at a distal end of the body portion and configured to be axially movable relative to a docking device for a prosthetic implant,
wherein the body portion comprises a lumen configured to receive the docking device therein,
wherein the tip portion comprises one or more slits defining one or more flaps, wherein the one or more flaps are movable between a radially collapsed state and a radially expanded state, wherein in the radially collapsed state, the one or more flaps cover a distal end of the docking device and occlude the lumen of the body portion, and wherein in the radially expanded state, the one or more flaps allow the distal end of the docking device to extend distally from the lumen of the body portion and beyond the tip portion such that the distal end of the docking device is uncovered by the dock sleeve.

2. The delivery apparatus of claim 1, wherein the tip portion comprises one slit dividing the tip portion into two flaps.

3. The delivery apparatus of claim 1, wherein the tip portion comprises two crossing slits dividing the tip portion into four flaps.

4. The delivery apparatus of claim 1, wherein the tip portion comprises an aperture located along a longitudinal axis of the dock sleeve.

5. The delivery apparatus of claim 1, wherein the tip portion comprises a proximal end connected to the distal end of the body portion and a distal end located distal to the distal end of the body portion, wherein the tip portion tapers radially inwardly from the proximal end of the tip portion to the distal end of the tip portion.

6. The delivery apparatus of claim 5, wherein a cross-sectional profile of the tip portion taken along a longitudinal axis of the dock sleeve forms a rounded shape between the proximal end of the tip portion and the distal end of the tip portion.

7. The delivery apparatus of claim 1, wherein the dock sleeve comprises one or more radiopaque markers, wherein at least one radiopaque marker is disposed on the tip portion.

8. The delivery apparatus of claim 7, wherein the at least one radiopaque marker is disposed at a distal-most area of the tip portion.

9. The delivery apparatus of claim 7, wherein the at least one radiopaque marker covers an entire area of the tip portion.

10. The delivery apparatus of claim 1, wherein an outer surface of the body portion and an outer surface of the tip portion comprise a hydrophilic coating.

11. The delivery apparatus of claim 10, wherein the hydrophilic coating comprises hydrogel.

12. A dock sleeve for implanting a docking device at a native valve, the dock sleeve comprising:
a body portion and a tip portion located at a distal end of the body portion,
wherein the dock sleeve is configured to be movable between a covered state and an uncovered state,
wherein when the dock sleeve is in the covered state, the body portion covers at least a distal portion of the docking device and the tip portion covers a distal end of the docking device,
wherein when the dock sleeve is in the uncovered state, the distal end of the docking device extends out of the dock sleeve through the tip portion of the dock sleeve.

13. A method of creating a dock sleeve configured to hold a docking device, the method comprising:
creating a dock sleeve comprising a body portion and a tip portion, wherein the tip portion completely closes a distal end of the body portion;
adding a coating material to the dock sleeve; and
creating at least one slit on the tip portion.

14. The method of claim 13, wherein adding the coating material to the dock sleeve comprises coating at least a portion of an outer surface of the body portion and an outer surface of the tip portion with the coating material.

15. The method of claim 13, wherein adding the coating material to the dock sleeve comprises dipping the dock sleeve to a solution of the coating material.

16. The method of claim 13, wherein creating the at least one slit on the tip portion comprises cutting open along a diameter of the tip portion.

17. The method of claim 13, wherein the at least one slit is a first slit, wherein the method further comprises cutting a second slit intersecting the first slit.

18. The method of claim 13, further comprising creating an aperture at a center of the tip portion.

19. The method of claim 13, wherein creating the dock sleeve comprises disposing one or more radiopaque markers on the dock sleeve.

20. The method of claim 19, wherein disposing the one or more radiopaque markers comprises disposing at least one radiopaque marker on the tip portion.
